(19) United States
(12) Patent Application Publication
(10) Pub. No.: US 2025/0361307 A1
(43) Pub. Date: Nov. 27, 2025

(54) COMBINATION THERAPIES FOR TREATING HYPERPROLIFERATIVE DISORDERS

(71) Applicant: X4 Pharmaceuticals, Inc., Boston, MA (US)

(72) Inventors: Chi Nguyen, Vienna (AT); Art Taveras, Boston, MA (US)

(21) Appl. No.: 18/873,661

(22) PCT Filed: Jun. 9, 2023
(86) PCT No.: PCT/US23/68229

§ 371 Date: Dec. 10, 2024

Related U.S. Application Data
(60) Provisional application No. 63/366,226, filed on Jun. 10, 2022.

(51) Int. Cl. C07K 16/28; A61K 31/437; A61K 31/4545; A61K 31/497; A61K 31/519; A61K 45/06; A61P 35/00

(52) U.S. Cl. CPC C07K 16/2866; A61K 31/437; A61K 31/4545; A61K 31/497; A61K 31/519; A61K 45/06; A61P 35/00

(57) ABSTRACT

The present invention provides methods of treating B-cell disorders with combination therapies comprising the use of (i) a targeted B-cell therapy; (ii) an IL-6 modulator, and (iii) a CXCR4 inhibitor; or (i) a targeted B-cell therapy and (II) an IL-6 modulator. In one aspect, the present invention provides a method of treating a hyperproliferative disorder in a patient in need thereof, comprising administering to the patient an effective amount of a targeted B-cell therapy in combination with an effective amount of an IL-6 modulator, and optionally in combination with an effective amount of a CXCR4 inhibitor.

### CROSS-REFERENCE TO RELATED APPLICATIONS

The present application claims the benefit of U.S. Provisional Application No. 63/366,226, filed Jun. 10, 2022; the entirety of which is hereby incorporated by reference.

### FIELD OF THE INVENTION

The present invention provides methods of treating hyperproliferative disorders with combination therapies comprising administering to a patient in need thereof a targeted B-cell therapy such as a BTK inhibitor, a BTK degrader, a BCL-2 inhibitor, a BH3 mimetic, or a proteasome inhibitor, in combination with an IL-6 modulator, and optionally in combination with a CXCR4 inhibitor.

### BACKGROUND OF THE INVENTION

Hyperproliferative disorders, including B-cell disorders, can be difficult to treat. For example, there will be approximately 80,500 new cases of non-Hodgkin's lymphomas diagnosed in 2022, which account for 4.2% of all new cancer cases. These patients have a five-year survival rate that is less than 75%. There will be 20,000 new cases of chronic lymphocytic leukemia (CLL) diagnosed in 2022, with a 5-year survival rate of about 88%. Thus, there remains a need for new and effective therapies, including combination therapies, to treat B-cell disorders such as non-Hodgkin's lymphoma. See seer.cancer.gov/statfacts/html/nhl.html.

Waldenström's macroglobulinemia (WM) is an indolent B-cell lymphoma characterized by accumulation of malignant lymphoplasmacytic cells in the bone marrow (BM). High levels of monoclonal immunoglobulin M (IgM) are secreted by WM cells, resulting in anemia, blood hyperviscosity syndrome, visual impairments, and neurological symptoms. Consequently, lowering serum IgM is a key end point in WM therapy and a common parameter to assess the success of any WM treatment.

Over the last decade, significant progress has been made in understanding the genetics underlying the pathogenesis of WM. Somatic mutations in clonal populations of cells lead to WM. The somatic L265P mutation in MYD88 innate immune signal transduction adaptor (MYD88) gene can be found in >90% of patients with WM. The MYD88 gene encodes a protein that is involved in signaling pathways, including activation of nuclear factor-kB upon stimulation of toll-like receptors (TLRs). Additionally, MYD88 anchors with phosphorylated Bruton tyrosine kinase (BTK), which itself is part of many signaling pathways, including toll-like, chemokine and B-cell receptors. MYD88^{L265P} is thought to be an activating mutation that increases binding to BTK, promoting cell survival and proliferation.

A second, more diverse category of mutation in WM, detected in approximately 30% of patients, can be found in the gene encoding C-X-C chemokine receptor 4 (CXCR4). The G protein-coupled receptor CXCR4 binds its natural ligand C-X-C chemokine ligand 12 (CXCL12), which is produced by the perivascular cells of the bone marrow stroma. Upon binding to CXCR4, CXCL12 induces downstream signaling activation of phosphoinositide 3-kinase (PI3K), which controls lymphocyte trafficking, chemotaxis, and cell survival. In WM, CXCR4 mutation generally occurs in the C terminal, intracellular domain of the protein—a region involved in signal transduction. Most CXCR4 C-terminal mutations found in WM cause hyperactivation of the receptor and its downstream signaling pathways, resulting in decreased internalization of the receptor and increased chemotaxis. Patients with MYD88^{L265P} CXCR4^{Mut} WM typically present with higher serum IgM levels and greater BM involvement compared with those with MYD881265P mutation alone.

IL6 is known to have a role in lymphoma. For example, PEL or body cavity-based lymphoma (BCBL) is an aggressive immunoblastic B cell malignancy which usually presents as an effusion in the body cavities of patients with acquired immunodeficiency syndrome. PEL cells constitutively produce IL-6 and express the IL-6R, and cell growth was inhibited by human IL-6 antisense oligonucleotides. An implication of IL-6 in the pathophysiology of a variety of B-cell leukemias and lymphomas as well as some non-B cell malignancies has been suggested. In many cases, serum IL-6 or sIL-6R levels are elevated, as shown for low and high-grade non-Hodgkin's lymphomas (NHL), Hodgkin's disease (HD), and in adult T cell leukemia/lymphoma.

In B cell chronic lymphocytic leukemia (B-CLL), the most common leukemia, the leukemic cells can produce IL-6, and in a subset of patients, IL-6 serum levels are elevated and correlate with disease stage and shorter survival rates. Serum sIL-6R levels also have prognostic value. In diffuse large B cell lymphoma (DLBCL), serum IL-6 levels correlate with prognosis and autocrine IL-6 production may provide proliferative and anti-apoptotic signals. In an analysis of IL-6 expression in high-grade B cell lymphomas comprising Burkitt's lymphomas (BL), DLBCL and immunoblastic lymphomas, IL-6 was mainly produced in tumor samples of non-BLs, but not in BLs. In mantle cell lymphoma, another aggressive B cell NHL, IL-6 was identified as a key growth and survival factor acting in an autocrine fashion. See Transfus Med Hemother 2013; 40:336-343, doi.org/10.1159/000354194. Moreover, IL-6 can induce therapeutic resistance for several cancer agents currently used to treat classical Hodgkin lymphoma (cHL), as shown using immunohistochemistry with an IL-6 antibody on tissue microarrays from diagnostic biopsies of CHL patients. See Blood Adv. 2021 Mar. 23; 5 (6): 1671-1681, doi: 10.1182/bloodadvances.2020003664.

### SUMMARY OF THE INVENTION

In one aspect, the present invention provides a method of treating a hyperproliferative disorder in a patient in need thereof, comprising administering to the patient an effective amount of a targeted B-cell therapy in combination with an effective amount of an IL-6 modulator, and optionally in combination with an effective amount of a CXCR4 inhibitor.

In some embodiments, the hyperproliferative disorder is a B-cell disorder.

In one aspect, the present invention provides a method of treating a hyperproliferative disorder in a patient in need thereof, comprising administering to the patient an effective amount of a targeted B-cell therapy in combination with an effective amount of an IL-6 modulator, and further in combination with an effective amount of a CXCR4 inhibitor.

In another aspect, the present invention provides a method of treating a hyperproliferative disorder in a patient in need thereof, comprising administering to the patient an effective amount of a targeted B-cell therapy and an effective amount of an IL-6 modulator, and, optionally, an effective amount of a CXCR4 inhibitor.

In another aspect, the present invention provides a method of treating a hyperproliferative disorder in a patient in need thereof, comprising administering to the patient an effective amount of a targeted B-cell therapy, an effective amount of an IL-6 modulator, and an effective amount of a CXCR4 inhibitor; wherein the doses of each are provided herein.

In another aspect, the present invention provides a method of treating a hyperproliferative disorder in a patient in need thereof, comprising administering to the patient effective amounts of one or more targeted B-cell therapies and an effective amount of CXCR4 inhibitor. In some embodiments, the targeted B-cell therapies are selected from BTK inhibitors, BCL-2 inhibitors/BH3 mimetics, and proteasome inhibitors, or pharmaceutically acceptable salts thereof.

In some embodiments, the targeted B-cell therapy is selected from a BTK inhibitor, a BCL-2 inhibitor/BH3 mimetic, and a proteasome inhibitor, or a pharmaceutically acceptable salt thereof.

In some embodiments, the IL-6 modulator is an IL-6 inhibitor. In some embodiments, the IL-6 modulator is an IL-6 receptor modulator. In some embodiments, the IL-6 receptor modulator is an IL-6 receptor antibody.

### BRIEF DESCRIPTION OF THE FIGURES

FIG. 1: BMSC-derived IL-6 causes IgM hypersecretion in WM cells via IL-6R-JAK-STAT3. IgM (A) and IL-6 (B) were measured in the supernatants of HS-27A BMSCs cocultured for 72 hours with MWCL-1 cells. Viability (C), pathway activation (D), and IgM secretion (E) in starved MWCL-1 cells treated with IL-6 were measured by CellTiter-Glo, phosphoflow, and ELISA, respectively. Relative IgM release in the presence of exogenous IL-6+/−tocilizumab (IL-6R antibody), PF-06263276 (pan-JAK inhibitor), or BP-1-102 (STAT3 inhibitor) was also measured (F). BMSC, bone marrow stromal cells; Ig, immunoglobulin; IL, interleukin; p-AKT, phosphor-PI3K-Akt; p-IKb, phosphor-nuclear factor of kappa light polypeptide gene enhancer in C-cells, inhibitor, alpha; p-JNK, phosphor-janus kinase; p-MAPK, phosphor-mitogen-activated protein kinase; p-STAT, signal transducer and activator of transcription; WM, Waldenström's Macroglobulinemia. In this and other figures, P values <0.05 were considered statistically significant and set as follows: **-P<0.01; ***-P<0.001; ****-P<0.0001.

FIG. 2: BMSC-derived IL-6 increases CXCR4 cell surface expression in WM cells via IL-6R-JAK-STAT3 signaling and enhances WM cell adhesion to BMSCs. Expression of CXCR4 was analyzed in publicly available gene expression data sets GSE171739 and GSE9656 (A). MWCL-1 cells were cocultured with HS-27A BMSCs+/−tocilizumab (B) or pretreated with tocilizumab, BP-1-102, or PF-06263276 (C) and CXCR4 cell surface expression measured via flow cytometry. The effects of IL-6 pretreatment on BMSC adhesion of MWCL-1 cells to HS-27A cells (D) was visualized using Calcein AM. BMSC, bone marrow stromal cells; CXCR4, C-X-C chemokine receptor 4; IL, interleukin; FDR, false discovery rate; WM, Waldenström's Macroglobulinemia.

FIG. 3: Mavorixafor causes disruption of WM cell migration and adhesion to BMSCs. The effects of mavorixafor pretreatment on BMSC adhesion to MWCL-1 cells cocultured with HS-27A BMSCs were visualized using Calcein AM (A). Migration of MWCL-1 cells toward CXCL12 with and without pretreatment with mavorixafor (B) and/or with and without HS-27A BMSCs coculture (C) was also measured by transwell migration assay. The effects of mavorixafor pretreatment on CXCL12-induced Ca²⁺ mobilization in MWCL-1 cells cocultured with HS-27A BMSCs were measured via Fluo-4 AM fluorescence (D). BMSC, bone marrow stromal cells; CXCR4, C-X-C chemokine receptor 4; CXCL12, C-X-C chemokine ligand 12; IL, interleukin; WM, Waldenström's Macroglobulinemia.

FIG. 4: Mavorixafor enhances antitumor activity of B-cell-targeted therapies in WM cells. Apoptosis of MWCL-1 cells treated with mavorixafor in combination with B-cell-targeted inhibitors was measured via flow cytometry (A-F). Synergistic activity between mavorixafor and B-cell-targeted inhibitors was analyzed via Chou and Talalay analysis (A-F). Cleavage of apoptotic markers in the presence of mavorixafor and ibrutinib was measured via immunoblot (G). CF, cytoplasmic fraction; MAV, mavorixafor; PARP, poly [ADP-ribose] polymerase.

FIG. 5: Mavorixafor overcomes BMSC-induced drug resistance. Apoptosis of MWCL-1 cells treated with mavorixafor in combination with B-cell-targeted inhibitors in MWC-1 cells/HS-27A BMSCs coculture was measured via flow cytometry (A-F). Cleavage of apoptotic markers in the presence of mavorixafor and ibrutinib was measured via immunoblot (G). BMSC, bone marrow stromal cells; CF, cytoplasmic fraction; CXCR4, C-X-C chemokine receptor 4; Evo, evobrutinib; Ibr, ibrutinib; Il, interleukin; PARP-1, poly [ADP-ribose] polymerase 1; Pir, pirtobrutinib; Mav, mavorixafor; Nem, nemtabrutinib; NS, not significant; Ven, venetoclax; WM, Waldenström's Macroglobulinemia.

FIG. 6: Mavorixafor as a single agent or in combination with B-cell-targeted therapies inhibited BMSC-induced IgM hypersecretion. MWCL-1 cells were preincubated with mavorixafor, B-cell-targeted inhibitors, or both, and cocultured with or without HS-27A BMSCs, followed by supernatant IgM measurements after 48 or 72 hours (A-G). BMSC, bone marrow stromal cells; CXCR4, C-X-C chemokine receptor 4; Evo, evobrutinib; Ibr, ibrutinib; Ig, immunoglobulin; Il, interleukin; PARP-1, poly [ADP-ribose] polymerase 1; Pir, pirtobrutinib; Mav, mavorixafor; Nem, nemtabrutinib; NS, not significant; Ven, venetoclax; WM, Waldenström's Macroglobulinemia.

FIG. 7: Viability of WM cells in the presence of IL-6R-JAK-STAT3 signaling inhibitors. Relative viability of MWCL-1 cells in the presence of tocilizumab (IL-6R antibody), BP-1-102 (STAT3 inhibitor), or PF-06263276 (pan-janus kinase inhibitor).

FIG. 8: BMSC-induced resistance of WM cells to B-cell-targeted therapies. Apoptosis and viability of MWCL-1 cells with and without coculture with HS-27A BMSCs in the presence of B-cell-targeted inhibitors (A-F).

FIG. 9: BMSC-induced IgM secretion by WM cells treated with B-cell-targeted therapies. IgM secretion by MWCL-1 cells with and without coculture with HS-27A BMSCs in the presence of B-cell-targeted inhibitors (A-F).

FIG. 10: HS-5 BMSCs reduced sensitivity of WM cells to B-cell-targeted therapies. Apoptosis of MWCL-1 cells (A,B) and IgM secretion by MWCL-1 cells (C,D) with and without coculture with HS-5 BMSCs in the presence of B-cell-targeted inhibitors, ibrutinib and zanubrutinib.

FIG. 11: Effect of mavorixafor on apoptosis of BMSCs in coculture with WM cells. Apoptosis of HS-27A (A) and HS-5 (B) BMSCs cocultured with WM cells in the presence of mavorixafor.

FIG. 12: IL-6 release in WM/BMSC coculture model. Effects of mavorixafor on IL-6 release in WM/BMSC coculture model.

FIG. 13A: Double and Triple Combination Therapies Described Herein Increase % Apoptotic Cell in WM (MWCL-1) cells in a co-culture model of WM (MWCL-1)-BMSC (HS-27a).

FIG. 13B: Double and Triple Combination Therapies Described Herein Decrease % IgM change in WM (MWCL-1) cells in a co-culture model of a WM (MWCL-19-BMSC (HS27a).

FIG. 14: Even in Absence of BMSC, Soluble IL-6 Upregulates CXCR4 Expression & IgM Secretion in Waldenström's macroglobulinemia (WM) Cells; which is Prevented by Blockade of IL-6/IL6R/STAT3 Axis.

FIG. 15. Ixazomib Kills Waldenström's Macroglobulinemia (WM; MWCL-1; MYD88^{L265P}-CXCR4^{WT}) Tumor Cells (in the absence of Bone Marrow Stroma). Mavorixafor Enhances Apoptosis Efficacy of Ixazomib. Time frame of assay=72 hours.

FIG. 16. Mavorixafor Synergizes with Ixazomib to Inhibit BM Stroma-Induced IgM Hypersecretion in Waldenström's Macroglobulinemia (WM; MWCL-1; MYD88^{L265P}-CXCR4^{WT}) Cells. BMSC: HS-27A cells. Time frame of assay=72 hours.

FIG. 17. Triple Combination of Venetoclax, Ibrutinib and Mavorixafor Enhances Apoptosis of Waldenström's Macroglobulinemia (WM; MWCL-1; MYD88^{L265P}-CXCR4^{WT}) Cells. BMSC: HS-27A cells. Time frame of assay=48 hours.

### DETAILED DESCRIPTION OF CERTAIN EMBODIMENTS OF THE INVENTION

In one aspect, the present invention provides a method of treating a hyperproliferative disorder, comprising administering to a patient in need thereof a targeted B-cell therapy such as a BTK inhibitor, a BTK degrader, a BCL-2 inhibitor, a BH3 mimetic, or a proteasome inhibitor, in combination with an IL-6 modulator, and optionally in combination with a CXCR4 inhibitor.

In one aspect, the present invention provides methods of treating a hyperproliferative disorder, comprising administering to a patient in need thereof a targeted B-cell therapy such as a BTK inhibitor, a BTK degrader, a BCL-2 inhibitor, a BH3 mimetic, or a proteasome inhibitor, in combination with an IL-6 modulator, and further in combination with a CXCR4 inhibitor.

### B-Cell Disorders

In some embodiments, the hyperproliferative disorder is selected from B-cell disorders; related lymphomas and leukemias including: non-Hodgkin's lymphomas, chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma.

In some embodiments, the hyperproliferative disorder is a B-cell disorder.

In some embodiments, the hyperproliferative disorder is selected from a lymphoma and a leukemia.

In some embodiments, the hyperproliferative disorder is selected from adenocarcinoma (lungs, pancreas, gastrointestinal, kidney) urogenital carcinoma, melanoma, glioblastoma, breast neoplasm, prostate cancer, primary central nervous system lymphoma, lymphoplasmacytic lymphoma, multiple myeloma, mantle cell lymphoma, T-cell leukemia/lymphoma, Karposi's Sarcoma, and Hodgkin's lymphoma.

In some embodiments, the B-cell disorder is selected from diffuse large B-cell lymphoma (DLBCL), primary mediastinal B-cell lymphoma, follicular lymphoma, chronic lymphocytic leukemia (CLL), small lymphocytic lymphoma (SLL), mantle cell lymphoma (MCL), marginal zone lymphomas (including extranodal marginal zone B-cell lymphoma, also known as mucosa-associated lymphoid tissue (MALT) lymphoma; nodal marginal zone B-cell lymphoma; and splenic marginal zone B-cell lymphoma), Burkitt lymphoma, Burkitt-like lymphoma, Waldenström's macroglobulinemia (WM), hairy cell leukemia, primary central nervous system lymphoma (PCNSL), and primary intraocular lymphoma.

In some embodiments, the B-cell disorder is an aggressive non-Hodgkin's lymphoma selected from diffuse large B-cell lymphoma (DLBCL), anaplastic large-cell lymphoma, Burkitt lymphoma, lymphoblastic lymphoma, mantle cell lymphoma, and peripheral t-cell lymphoma.

In some embodiments, the B-cell disorder is an indolent Non-Hodgkin's lymphoma selected from follicular lymphoma, cutaneous T-cell lymphoma, lymphoplasmacytic lymphoma marginal zone B-cell lymphoma, mucosa-associated lymphoid tissue (MALT) lymphoma, and small-cell lymphocytic lymphoma.

In some embodiments, the B-cell disorder is selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma.

In some embodiments, the B-cell disorder is Waldenström's macroglobulinemia (WM).

### First Agent: A Targeted B-Cell Therapy

The first agent in the combination therapies described herein comprises a targeted B-cell therapy. In some embodiments, the targeted B-cell therapy is selected from a BTK inhibitor, a BTK degrader, a BCL-2 inhibitor/BH3 mimetic, and a proteasome inhibitor, or a pharmaceutically acceptable salt thereof.

### BTK Inhibitors

In some embodiments, the targeted B-cell therapy is a BTK inhibitor or a pharmaceutically acceptable salt thereof.

In some embodiments, the BTK inhibitor is selected from Ibrutinib (Imbruvica® Abb Vie); Zanubritinib (Brukinsa® BeiGene); Acalubritinib (Calquence® AstraZeneca Pharmaceuticals); Evobrutinib (Merck KgA); Tirabrutinib (Velexbru®, Ono Pharmaceuticals; Gilead Sciences); Rilzabrutinib (PRN-1008; Principia; Sanofi); Tolebrutinib (PRN-2246; SAR442168; Principia; Sanofi); Fenebrutinib (GDC-0853) Genentech; Orelabrutinib (ICP-022; Innocare Pharma); Branebrutinib, BMS-986195 (Bristol Myers Squibb); Elsubrutinib, ABBV-105 (Abbvie); Remibrutinib, LOU064 (Novartis); Spebrutinib (CC-292 AVL-292; Avila/Celgene); Poseltinib (HM71035/LSN3359180; Hammi (Korea)/Lilly); vecabrutinib, LCB 03-0110; LFM-A13; PCI 29732; PF 06465469; (−)-Terreic acid; BMX-IN-1; BI-BTK-1; BMS-986142; CGI-1746; GDC-0834; olmutinib, PLS-123; PRN1008; RN-486; Nemtabrutinib (ARQ-531, MK1026) (Merck); and Pirtobrutinib (LOXO-305) (Lilly).

In some embodiments, the BTK inhibitor is selected from Ibrutinib; Zanubritinib; Acalubritinib; Evobrutinib; ARQ-531OXO-305; tirabrutinib, fenebrutinib, poseltinib, vecabrutinib, spebrutinib, LCB 03-0110, LFM-A13, PCI 29732, PF 06465469, (−)-Terreic acid, BMX-IN-1, BI-BTK-1, BMS-986142, CGI-1746, GDC-0834, olmutinib, PLS-123, PRN1008, and RN-486.

In some embodiments, the BTK inhibitor is selected from ibrutinib, zanubritinib, acalubritinib, evobrutinib, ARQ-531, and OXO-305.

In other embodiments, the BTK inhibitor is selected from ibrutinib, acalabrutinib, zanubrutinib, tirabrutinib, evobrutinib, fenebrutinib, poseltinib, vecabrutinib, spebrutinib, LCB 03-0110, LFM-A13, PCI 29732, PF 06465469, (−)-Terreic acid, BMX-IN-1, BI-BTK-1, BMS-986142, CGI-1746, GDC-0834, olmutinib, PLS-123, PRN1008, RN-486, LOXO-305 (pirtobrutinib), and ARQ-531 (nemtabrutinib; MK-1026); or a pharmaceutically acceptable salt thereof.

In some embodiments, the BTK inhibitor is selected from ibrutinib, tirabrutinib, evobrutinib, fenebrutinib, poseltinib, vecabrutinib, spebrutinib, LCB 03-0110, LFM-A13, PCI 29732, PF 06465469, (−)-Terreic acid, BMX-IN-1, BI-BTK-1, BMS-986142, CGI-1746, GDC-0834, olmutinib, PLS-123, PRN1008, RN-486, LOXO-305 (pirtobrutinib), and ARQ-531 (nemtabrutinib; MK-1026); or a pharmaceutically acceptable salt thereof.

In some embodiments, the BTK inhibitor is selected from ibrutinib, evobrutinib, LOXO-305, and ARQ-531, or a pharmaceutically acceptable salt thereof.

In some embodiments, the BTK inhibitor is ibrutinib, or a pharmaceutically acceptable salt thereof.

### BTK Degraders

Degradation of Bruton's tyrosine kinase mutants by proteolysis-targeting chimera (PROTAC) for a potential treatment of ibrutinib-resistant non-Hodgkin's lymphomas has been reported. PROTAC is a novel strategy for the selective knockdown of target proteins by small molecules, which utilizes the ubiquitin-protease system to target a specific protein and induce its degradation in the cell. The ubiquitin-protease system (UPS), also known as the ubiquitin-proteasome pathway (UPP), is a common post-translational regulation mechanism that is responsible for protein degradation in normal and pathological states. Ubiquitin, which is highly conserved in eukaryotic cells, is a modifier molecule, composed of 76 amino acids, that covalently binds to and labels target substrates via a cascade of enzymatic reactions involving E1, E2, and E3 enzymes. Subsequently, the modified substrate is recognized by the 26S proteasome complex for ubiquitination-mediated degradation. Two E1 enzymes have been discovered, whereas ˜40 E2 enzymes and more than 600 E3 enzymes offer the functional diversity to govern the activity of many downstream protein substrates. A limited number of E3 ubiquitin ligases have been successfully hijacked for use by small-molecule PROTAC technology, including the Von Hippel-Lindau disease tumor suppressor protein (VHL), the Mouse Double Minute 2 homologue (MDM2), the Cellular Inhibitor of Apoptosis (cIAP), and cereblon.

As explored in a recent Phase 1 trial by Nurix Pharmaceuticals, NX-2127, a novel orally bioavailable degrader of the Bruton tyrosine kinase (BTK), demonstrated clinically meaningful degradation of the BTK in patients with relapsed/refractory chronic lymphocytic leukemia (CLL) and other B-cell disorders. NX-2127 carries the normal cellular protein degradation mechanism which allows it to catalyze degradation of BTK. This mechanism is important in B-cell disorders because the BTK enzyme is present in the B-cell development, differentiation, and signaling that helps lymphoma and leukemia cells survive. The phase 1 clinical trial was designed to investigate the safety and tolerability of NX-2127 in patients with B-cell disorders, including CLL, small lymphocytic lymphoma (SLL), mantle cell lymphoma, marginal zone lymphoma, Waldenström's macroglobulinemia (WM), follicular lymphoma, and diffuse large B-cell lymphoma.

Accordingly, BTK degraders such as NX-2127, MT802, L18I, SPB5208, or RC-1 may be used in the present invention. See, e.g., Yu, F., et al., Front. Chem., 30 Jun. 2021, which is hereby incorporated by reference.

### BCL-2 Inhibitor or BH3 Mimetic

In some embodiments, the targeted B-cell therapy is a BCL-2 inhibitor or a BH3 mimetic, or a pharmaceutically acceptable salt thereof.

The BCL-2 protein is the founding member of the BCL-2 family of apoptosis regulators and was the first apoptosis modulator to be associated with cancer. The recognition of the important role played by BCL-2 for cancer development and resistance to treatment made it a relevant target for therapy for many diseases, including solid tumors and hematological neoplasias. Among the different strategies that have been developed to inhibit BCL-2, BH3-mimetics have emerged as a novel class of compounds with favorable results in different clinical settings, including chronic lymphocytic leukemia (CLL). Venetoclax (also known as ABT-199), a potent and selective inhibitor of BCL-2, was approved by the FDA in 2016 for treatment of relapsed/refractory chronic lymphocytic leukemia (CLL) with 17p deletion based on its favorable safety profile and high response rates. The BCL-2 family's members of this family can be grouped in three main categories. The anti-apoptotic subfamily is characterized by the presence of four BCL-2 homology (BH) domains (BH1, BH2, BH3, and BH4) and, in humans, includes the proteins BCL-2 (the founding member), BCL-XL, BCL-w, BCL-2-related protein A1 (Bf1-1/A1), myeloid cell leukemia 1 (MCL-1), and BCLB/Boo. The pro-apoptotic members can be divided in two subfamilies: the multi-domain pro-apoptotic ‘effectors’ (such as BAK and BAX) and those members known as ‘BH3-only proteins’ as they only have the short BH3 domain. The latter subfamily includes BAD, BID, BIK, BIM, BMF, HRK, PUMA, and NOXA.

High levels of BCL-2 are observed in patients with FL, CLL, mantle-cell lymphoma (MCL), and Waldenström's macroglobulinemia. A heterogeneous pattern of expression of BCL-2 is reported among other hematological neoplasms, such as diffuse large B-cell lymphoma (DLBCL), for which certain subtypes present low levels of this molecule; and multiple myeloma (MM), in which BCL-2 expression is especially elevated in patients harboring t(11;14).

The development of selective inhibitors for BCL-2 and BCL-XL is limited by the high degree of similarity shared by their BH3 domain. Using reverse engineering, a new compound was developed to overcome the unfavorable effect of navitoclax on platelets as the consequence of BCL-XL inhibition, while keeping its anti-tumor activity. Venetoclax is a potent and selective inhibitor of the BCL-2 protein that has demonstrated clinical efficacy in several hematological malignancies. Contrasting with navitoclax, which targets BCL-2 and BCL-XL, venetoclax has a distinct mode of action as it binds and neutralizes BCL-2 with sub-nanomolar affinity (Ki<0.010 nM), while interacting only weakly with BCL-XL and BCL-W. By sparing BCL-XL, it exerts little effect on platelet numbers. In preclinical studies, this orally bioavailable inhibitor showed cell-killing activity against a variety of cell lines, including cell lines derived from ALL, NHL, and AML. When investigated in xenograft models using hematological tumors, venetoclax promoted tumor growth inhibition in a dose-dependent fashion. For those models in which venetoclax had little effect as single-agent, improved efficacy was achieved with the combination with other drugs. Venetoclax has been investigated for treatment of CLL and been tested in combination with numerous anticancer agents for cancers such as AML, MM, MCL, CLL/SLL, B-cell lymphoma, and DLBCL.

Exemplary BCL-2 inhibitors useful in the present invention include venetoclax (Velcade®) and navitoclax. Another useful BCL-2 inhibitor is AT-101. AT-101 is an orally active pan-Bcl-2 inhibitor that consists of gossypol, a natural compound derived from the cotton plant. AT-101 has shown potential efficacy in combinations with other drugs for treatment of solid tumors, such as in combination with docetaxel, topotecan, paclitaxel and carboplatin, cisplatin and etoposide. Other BCL-2 inhibitors include sabutoclax, S55746, HA-14-1 and gambogic acid (Han et al. (2019) BioMed Research International 2019: Article ID 1212369: Drugs and Clinical Approaches Targeting the Antiapoptotic Protein: A Review; hereby incorporated by reference).

In some embodiments, the targeted B-cell therapy is a BH3 mimetic or a pharmaceutically acceptable salt thereof.

BH3 mimetics comprise a novel class of BCL-2 inhibitors that have shown promising results in several hematological malignancies, both as single agents and in combination with other anti-cancer drugs. This novel class of compounds is designed to selectively kill cancer cells by targeting the mechanism involved in their survival. These agents induce apoptosis by mimicking the activity of natural antagonists of BCL-2 and other related proteins. For example, ABT-737, developed by Abbott Laboratories (North Chicago, IL, USA), is considered the prototype of BH3 mimetics as it was the first-in-class compound developed to mimic the function of BH3-only-proteins. Discovered using a high-throughput nuclear magnetic resonance-based screening method to identify small molecules that bind to the BH3-binding groove of BCL-XL, ABT-737 binds with a much higher affinity (<1 nmol/L) than previous compounds to anti-apoptotic proteins BCL-2, BCL-XL and BCL-w, blocking their function.

Navitoclax, a potent and selective inhibitor of BCL-2, is the second generation, orally bioavailable form of ABT-737. Like its predecessor, navitoclax interacts with high affinity and abrogates BCL-2, BCL-XL, and BCL-w, but has no activity against A1 and MCL-1. Navitoclax showed in vitro activity against a broad panel of tumor cell lines both as single agent and in combination with chemotherapy. In in vivo experiments, treatment with this inhibitor induced rapid and complete tumor responses in multiple xenograft models developed using small-cell lung cancer and hematologic cell lines, with responses lasting several weeks in some models. Moreover, in B-cell malignant xenograft models, cotreatment with navitoclax significantly improved the efficacy of numerous approved anti-cancer agents. Navitoclax potentiated the activity of rituximab in the B-cell lymphoma flank xenograft model, of modified R-CHOP regimen in a flank xenograft model of MCL, and of bortezomib in an MM model.

BH3 mimetics useful in the present invention include ABT-737, navitoclax, and obatoclax mesylate (GX15-070).

In some embodiments, the BCL-2 inhibitor/BH3 mimetic is selected from venetoclax (Venclexta® Abb Vie/Genentech), BGB-11417, LOXO-338, LP-108, S55746, APG-2575, APG-1252 (pelcitoclax), AT-101, TQB3909, obatoclax, GDC-0199, ABT-737, and navitoclax (ABT-263); or a pharmaceutically acceptable salt thereof.

In some embodiments, the BCL-2 inhibitor is venetoclax.

In some embodiments, the targeted B-cell therapies are a BCL-2 inhibitor and a BTK inhibitor.

In some embodiments, the targeted B-cell therapies are venetoclax and ibrutinib.

### Protease Inhibitor

In some embodiments, the targeted B-cell therapy is a proteasome inhibitor, or a pharmaceutically acceptable salt thereof.

Proteasome inhibitors useful in the present invention include ixazomib (Ninlaro®, Takeda); bortezomib (Velcade®; Millennium Pharmaceuticals; Takeda), carfilzomib (Kyprolis®; Amgen); thalidomide, and everolimus.

In some embodiments, the targeted B-cell therapy is ixazomib; bortezomib; or carfilzomib.

In some embodiments, the target B-cell therapy is ixazomib.

Proteasome inhibitors useful in the present invention include ixazomib (Ninlaro®), bortezomib (Velcade®), carfilzomib (Kyprolis®), marizomib (NPI-0052), oprozomib (ONX0912), ONX 0914 (an immunoproteasome selective inhibitor), and KZR-616 (an immunoproteasome inhibitor).

In some embodiments, the proteosome inhibitor is selected from Velcade® (bortezomib, Takeda Pharmaceuticals); Ninlaro® (ixazomib, Takeda Pharmaceuticals); Kyprolis® (carfilzomib, Onyx Pharmaceuticals Inc./Amgen); thalidomide, and everolimus.

Second Agent: Modulator of IL-6 and/or JAK/STAT3 Pathway

As described generally above, the second agent in the combination therapies disclosed herein is a modulator of IL-6 and/or JAK/STAT3 pathway. Any known IL-6 inhibitor or JAK/STAT3 inhibitor may be used in the combination therapy of this disclosure.

In some embodiments, the second agent is an IL-6 modulator.

In some embodiments, the IL-6 modulator is an IL-6 inhibitor. In some embodiments, the IL-6 modulator is an IL-6 antibody. In some embodiments, the IL-6 modulator is an IL-6 ligand antibody. In other words, the IL-6 modulator can act on the antibody or on the ligand.

In some embodiments, the IL-6 modulator is selected from tocilizumab (Actemra® Genentech), an IL-6r antibody; Sarilumab (Kevzara®, Sanofi/Regeneron), a recombinant humanized anti-IL-6R mAb; Satralizumab (Enspryng® Chugai and Roche), a humanized anti-IL-6R mAb; Siltuximab (SYLVANT®, EUSA Pharma), and IL-6 antagonist approved for treatment of patients with multicentric Castleman's disease (MCD); Vobarilizumab (ALX-0061 Ablynx), an investigational bispecific peptide nanobody which binds soluble IL-6R and human albumin; Olokizumab (CDP6038, UCB Pharma/R-Pharm); Sirukumab (CNTO-136; Johnson & Johnson); Clazakizumab (formerly ALD518 and BMS-945429), Ziltivekimab; (COR-001; Novo Nordisk); and Avidia-C326.

In some embodiments, the IL-6 modulator is selected from tocilizumab, siltuximab, sarilumab, olokizumab (CDP6038), elsilimomab, clazakizumab (BMS-945429, ALD518), sirukumab (CNTO 136), levilimab (BCD-089), CPSI-2364, ALX-0061, ARGX-109, FE301, and FM101.

In some embodiments, the IL-6 modulator is selected from tocilizumab; sarilumab; and satralizumab.

In some embodiments, the IL-6 modulator is tocilizumab (Actemra® Genentech).

In some embodiments, the second agent is a JAK/STAT3 inhibitor. The JAK/STAT3 inhibitor may directly or indirectly inhibit JAK, STAT3, or both.

In some embodiments, the JAK/STAT3 inhibitor is selected from aptamer-siRNA chimera; BBI608 (napabucasin); Celecoxib (Celebrex; Bextra); Pyrimethamine; C188-9; OPB-111077; OPB-31121; OPB-51602; Niclosamide; AZD-1480-Oligonucleotide; Ruxolitinib; Dasatinib; Siltuximab; BP-1-102; PF-06263276 (Pfizer); LLL12B, Tofacitinib, Baricitinib; Ruxolitinib; and Peficitinib.

### Third Agent: CXCR4 Inhibitor

In some embodiments, a CXCR4 inhibitor is co-administered in the combination therapies disclosed above. The CXCR4 may be administered prior to, concurrently with, or subsequent to administration of the targeted B-cell therapy and the IL-6 modulator.

In some embodiments, the CXCR4 inhibitor is selected from CXCR4 inhibitors disclosed in WO2017/223229 (including compounds I-1 through I-184 disclosed therein), WO2017/223239 (including compounds I-1 through I-229 disclosed therein), WO2017/223243 (including compounds I-1 through I-149 disclosed therein), WO2019/126106 (including compounds I-1 through I-69 disclosed therein), WO2020/264292 (including compounds I-1 through I-31 disclosed therein), and WO2021/263203 (including compounds I-1 through I-118 disclosed therein).

In other embodiments, the CXCR4 inhibitor is selected from the small molecule CXCR4 inhibitors disclosed in U.S. Pat. Nos. 7,291,631; 7,332,605; 7,354,932; 7,354,934; 7,501,518; 7,550,484; 7,723,525; 7,863,293; 8,778,967; 10,322,111; 7,414,065; 7,022,717; 7,084,155; 7,807,694; 6,750,348; 7,169,750; 7,491,735; and 7,790,747. The disclosures of the above documents are hereby specifically incorporated herein by reference.

In some embodiments, the CXCR4 inhibitor is selected from mavorixafor; plerixafor (AMD-3100; Sanofi); locuplumab (BMS-936564/MDX1338, Bristol Myers), a fully human anti-CXCR4 antibody, Kashyap et al. (2015) Oncotarget 7:2809-2822; Motixafortide (BL-8040; BKT-140; BiolineRx) Crees et al. (2021) Blood, 138 (Suppl): 475 Abstract 711; POL6326 (balixafortide, Polyphor) Karpova et al. (2017) Journal of Translational Medicine 15:2; PRX177561 (Proximagen) Gravina et al. (2017) Tumor Biology, June 2017:1-17; USL311 (Upsher-Smith Laboratories) NCT02765165; Burixafor hydrobromide (TG-0054, TaiGen Biotechnology) NCT02478125; LY2510924 (MEDI4736, Eli Lilly); NCT02737072, a CXCR4 Cyclic peptide antagonist; PF06747143 (Pfizer), a CXCR4 humanized IgG1 antagonist; BGB-11417; LOXO-338; LP-108; S55746; APG-257; APG-1252 (pelcitoclax); AT-101; TQB3909; obatoclax; GDC-0199; ABT-737; and navitoclax (ABT-263); or a pharmaceutically acceptable salt thereof.

In some embodiments, the CXCR4 inhibitor is mavorixafor, plerixafor, ulocuplumab, motixafortide, POL6326, PRX177561, USL311, burixafor (e.g., burixafor HBr), LY2510924, PF06747143, CX549, BPRCX807, TC14012, USL-311, FC131, CTCE-9908, or GMI 1359; or a pharmaceutically acceptable salt thereof.

In some embodiments, the CXCR4 inhibitor is selected from one of the following:

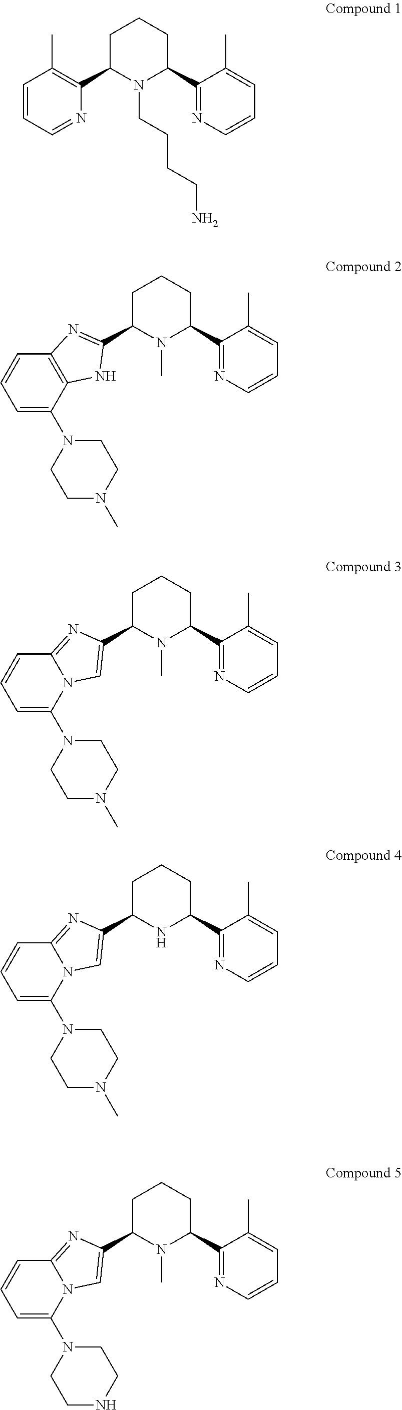 [SMILES: Cc1cccnc1[C@H]1CCC[C@@H](c2ncccc2C)N1CCCCN ; Cc1cccnc1[C@@H]1CCC[C@H](c2nc3cccc(N4CCN(C)CC4)c3[nH]2)N1C ; Cc1cccnc1[C@@H]1CCC[C@H](c2cn3c(N4CCN(C)CC4)cccc3n2)N1C ; Cc1cccnc1[C@@H]1CCC[C@H](c2cn3c(N4CCN(C)CC4)cccc3n2)N1 ; Cc1cccnc1[C@@H]1CCC[C@H](c2cn3c(N4CCNCC4)cccc3n2)N1C]

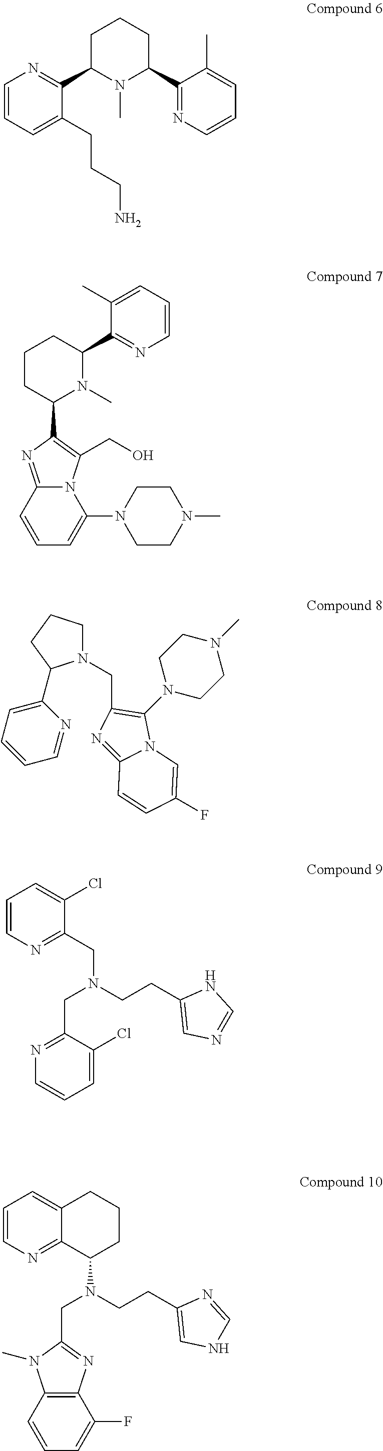

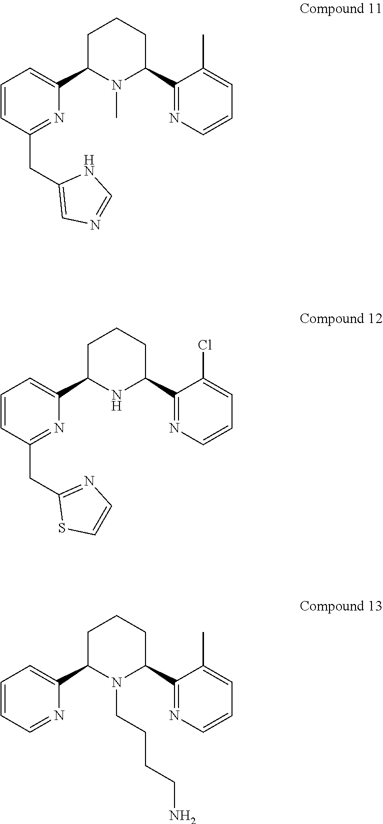 [SMILES: Cc1cccnc1[C@@H]1CCC[C@H](c2cccc(Cc3cnc[nH]3)n2)N1C ; Clc1cccnc1[C@@H]1CCC[C@H](c2cccc(Cc3nccs3)n2)N1 ; Cc1cccnc1[C@@H]1CCC[C@H](c2ccccn2)N1CCCCN]

or a pharmaceutically acceptable salt thereof.

### Exemplary Combination Treatments

In some embodiments, a method described herein relates to treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, or diffuse large B-cell lymphoma in a patient in need thereof comprises administering an effective amount of a first agent or a pharmaceutically acceptable salt thereof selected from Column 1 of Table 1, and administering an effective amount of a second agent or a pharmaceutically acceptable salt thereof selected from Column 2 of Table 1, and optionally, administering an effective amount of a third agent or a pharmaceutically acceptable salt thereof selected from Column 3 of Table 1:

TABLE 1
Combination Therapies for B-cell Disorders

| Column 1: | Column 2: | Column 3: |
|---|---|---|
| Targeted B-cell therapy | IL-6 modulator | CXCR4 inhibitor |
| Ibrutinib | Tocilizumab | Mavorixafor |
| Zanubritinib | Vobarilizumab | Plerixafor |
| Acalubritinib | Siltuximab | Ulocuplumab |
| Evobrutinib | Olokizumab | Motixafortide |
| Venetoclax | Sirukumab | POL6326 |
| Nemtabrutinib | Clazakizumab | PRX177561 |
| Pirtobrutinib | Ziltivekimab; | USL311 |
| Tirabrutinib | Avidia | Burixafor HBr |
| Fenebrutinib | | LY2510924 |
| Poseltinib | | PF06747143 |
| Vecabrutinib | | Compound 1 |
| Spebrutinib | | 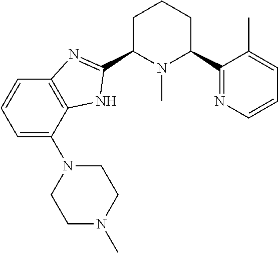 [SMILES: Cc1cccnc1[C@@H]1CCC[C@H](c2nc3cccc(N4CCN(C)CC4)c3[nH]2)N1C] Compound 2 |
| LCB 03-0110 | | 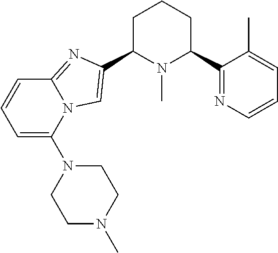 [SMILES: Cc1cccnc1[C@@H]1CCC[C@H](c2cn3c(N4CCN(C)CC4)cccc3n2)N1C] Compound 3 |
| LFM-A13 | | 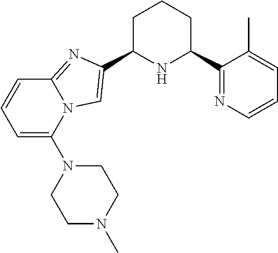 [SMILES: Cc1cccnc1[C@@H]1CCC[C@H](c2cn3c(N4CCN(C)CC4)cccc3n2)N1] Compound 4 |
| PCI 29732 | | 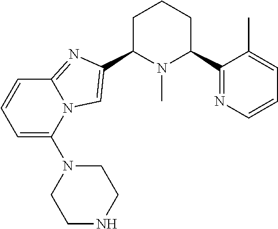 [SMILES: Cc1cccnc1[C@@H]1CCC[C@H](c2cn3c(N4CCNCC4)cccc3n2)N1C] Compound 5 |
| PF 06465469 | | 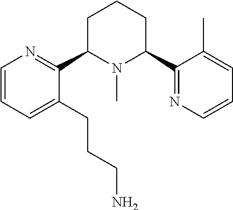 [SMILES: Cc1cccnc1[C@@H]1CCC[C@H](c2ncccc2CCCN)N1C] Compound 6 |
| (—)-Terreic acid, | | 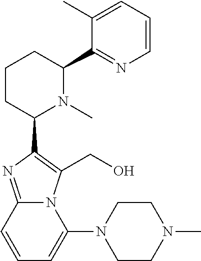 [SMILES: Cc1cccnc1[C@@H]1CCC[C@H](c2nc3cccc(N4CCN(C)CC4)n3c2CO)N1C] Compound 7 |
| BMX-IN-1 | | 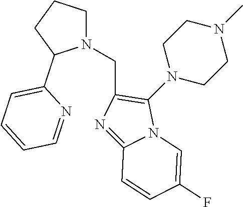 [SMILES: CN1CCN(c2c(CN3CCCC3c3ccccn3)nc3ccc(F)cn23)CC1] Compound 8 |
| BI-BTK-1 | | 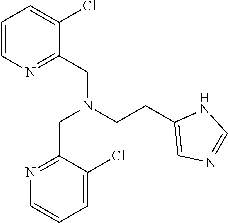 [SMILES: Clc1cccnc1CN(CCc1cnc[nH]1)Cc1ncccc1Cl] Compound 9 |
| BMS-986142 | | 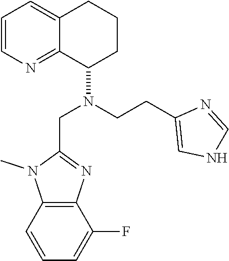 [SMILES: Cn1c(CN(CCc2c[nH]cn2)[C@H]2CCCc3cccnc32)nc2c(F)cccc21] Compound 10 |
| CGI-1746 | | 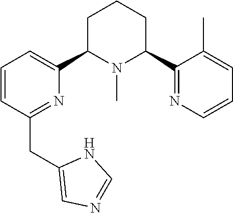 [SMILES: Cc1cccnc1[C@@H]1CCC[C@H](c2cccc(Cc3cnc[nH]3)n2)N1C] Compound 11 |
| GDC-0834 | | CX549 |
| Olmutinib | | BPRCX807 |
| PLS-123 | | TC14012 |
| PRN1008 | | USL-311 |
| RN-486 | | FC131 |
| pirtobrutinib | | CTCE-9908 |
| nemtabrutinib | | GMI 1359 |
| BGB-11417 | | 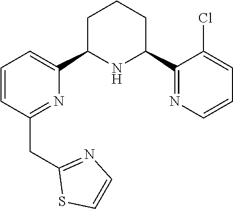 [SMILES: Clc1cccnc1[C@@H]1CCC[C@H](c2cccc(Cc3nccs3)n2)N1] Compound 12 |
| LOXO-338 | | 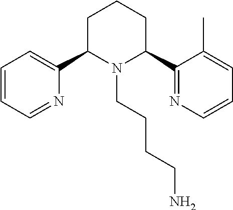 [SMILES: Cc1cccnc1[C@@H]1CCC[C@H](c2ccccn2)N1CCCCN] Compound 13 |
| LP-108 | | |
| S55746 | | |
| APG-2575 | | |
| Pelcitoclax | | |
| AT-101 | | |
| TQB3909 | | |
| obatoclax | | |
| GDC-0199 | | |
| ABT-737 | | |
| Navitoclax | | |
| Ixazomib | | |
| Bortezomib | | |
| Carfilzomib | | |
| Thalidomide | | |
| Everolimus | | |

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Tocilizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Tocilizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Sarilumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Sarilumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Satralizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Satralizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Vobarilizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Vobarilizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Siltuximab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Siltuximab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Olokizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Olokizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Sirukumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Sirukumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Clazakizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Clazakizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Ziltivekimab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Ziltivekimab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Avidia.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Avidia and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Tocilizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Tocilizumab and further in combination with an effective amount of

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Sarilumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Sarilumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Satralizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Satralizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Vobarilizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Vobarilizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Siltuximab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Siltuximab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinibor a pharmaceutically acceptable salt thereof in combination with an effective amount of Olokizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Olokizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Sirukumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Sirukumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Clazakizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Clazakizumab and further in combination with an effective amount of

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Ziltivekimab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Ziltivekimab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinibor a pharmaceutically acceptable salt thereof in combination with an effective amount of Avidia.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Zanubritinib or a pharmaceutically acceptable salt thereof in combination with an effective amount of Avidia and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Tocilizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Tocilizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Sarilumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Sarilumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Satralizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Satralizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Vobarilizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Vobarilizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Siltuximab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Siltuximab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Olokizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Olokizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Sirukumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Sirukumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Clazakizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Clazakizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Ziltivekimab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Ziltivekimab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Avidia.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Acalubritinib or a pharmaceutically acceptable salt thereof in combination with an effective amount of Avidia and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Tocilizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Tocilizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Sarilumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Sarilumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Satralizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Satralizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Vobarilizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Vobarilizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Siltuximab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Siltuximab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Olokizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Olokizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Sirukumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Sirukumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Clazakizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Clazakizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Ziltivekimab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Ziltivekimab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Avidia.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Ibrutinib or a pharmaceutically acceptable salt thereof in combination with an effective amount of Avidia and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Tocilizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Tocilizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Sarilumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Sarilumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Satralizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Satralizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Vobarilizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Vobarilizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Siltuximab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Siltuximab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Olokizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Olokizumab and further in combination with an effective amount of

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Sirukumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Sirukumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Clazakizumab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Clazakizumab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Ziltivekimab.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Ziltivekimab and further in combination with an effective amount of mavorixafor or a pharmaceutically acceptable salt thereof.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Avidia.

In some embodiments, methods of treating a B-cell disorder selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma in a patient in need thereof comprise administering an effective amount of Venetoclax or a pharmaceutically acceptable salt thereof in combination with an effective amount of Avidia and further in combination with an effective amount of mavorixafor or a pharmaceutically acceptable salt thereof.

### Dosage and Formulations

In another aspect, the present invention provides a method of treating a hyperproliferative disorder in a patient in need thereof, comprising administering to the patient an effective amount of a targeted B-cell therapy and an effective amount of an IL-6 modulator, and optionally an effective amount of a CXCR4 inhibitor; wherein the doses of each are provided herein.

In some embodiments, a targeted B-cell therapy is co-administered with an IL-6 modulator or a pharmaceutically acceptable salt thereof. In some embodiments, the targeted B cell therapy is ibrutinib, acalabrutinib, or zanubrutinib; or a pharmaceutically acceptable salt thereof.

In some embodiments, the present invention provides a method of treating a B-cell disorder in a patient in need thereof, as described herein, comprising administering to the patient a targeted B cell therapy in combination with one or more additional therapies wherein the combination of the targeted B-cell therapy and the one or more additional therapies acts synergistically. In some embodiments, the administration of the targeted B-cell therapy in combination with an additional therapeutic agent results in a reduction of the effective amount of that additional therapeutic agent as compared to the effective amount of the additional therapeutic agent in the absence of administration in combination with the targeted B-cell therapy. In some embodiments, the effective amount of the additional therapeutic agent administered in combination with the targeted B-cell therapy is about 90%, about 80%, about 70%, about 60%, about 50%, about 40%, about 30%, about 20%, or about 10% of the effective amount of the additional therapeutic agent in the absence of administration in combination with the targeted B-cell therapy.

In some embodiments, the targeted B-cell therapy or pharmaceutically acceptable salt thereof and additional therapy have a combination index (CI) of about 0.9 or less. In some embodiments, the targeted B-cell therapy or pharmaceutically acceptable salt thereof and additional therapy have a combination index (CI) of about 0.8 or less. In some embodiments, the targeted B-cell therapy or pharmaceutically acceptable salt thereof and additional therapy have a combination index (CI) of about 0.7 or less. In some embodiments, the targeted B-cell therapy or pharmaceutically acceptable salt thereof and additional therapy have a combination index (CI) of about 0.6 or less. In some embodiments, the targeted B-cell therapy or pharmaceutically acceptable salt thereof and additional therapy have a combination index (CI) of about 0.5 or less. In some embodiments, the targeted B-cell therapy or pharmaceutically acceptable salt thereof and additional therapy have a combination index (CI) of about 0.4 or less. In some embodiments, the targeted B-cell therapy or pharmaceutically acceptable salt thereof and additional therapy have a combination index (CI) of about 0.3 or less. In some embodiments, the targeted B-cell therapy or pharmaceutically acceptable salt thereof and additional therapy have a combination index (CI) of about 0.2 or less. In some embodiments, the targeted B-cell therapy or pharmaceutically acceptable salt thereof and additional therapy have a combination index (CI) of about 0.1 or less.

In some embodiments, the targeted B-cell therapy or pharmaceutically acceptable salt thereof and additional therapy have a combination index (CI) of about 0.1 to about 0.9. In some embodiments, the targeted B-cell therapy or pharmaceutically acceptable salt thereof and additional therapy have a combination index (CI) of about 0.1 to about 0.8. In some embodiments, the targeted B-cell therapy or pharmaceutically acceptable salt thereof and additional therapy have a combination index (CI) of about 0.1 to about 0.7. In some embodiments, the targeted B-cell therapy or pharmaceutically acceptable salt thereof and additional therapy have a combination index (CI) of about 0.1 to about 0.6. In some embodiments, the targeted B-cell therapy or pharmaceutically acceptable salt thereof and additional therapy have a combination index (CI) of about 0.1 to about 0.5. In some embodiments, the targeted B-cell therapy or pharmaceutically acceptable salt thereof and additional therapy have a combination index (CI) of about 0.1 to about 0.4. In some embodiments, the targeted B-cell therapy or pharmaceutically acceptable salt thereof and additional therapy have a combination index (CI) of about 0.1 to about 0.3. In some embodiments, the targeted B-cell therapy or pharmaceutically acceptable salt thereof and additional therapy have a combination index (CI) of about 0.1 to about 0.2. In some embodiments, the targeted B-cell therapy or pharmaceutically acceptable salt thereof and additional therapy have a combination index (CI) of about 0.2 to about 0.9. In some embodiments, the targeted B-cell therapy or pharmaceutically acceptable salt thereof and additional therapy have a combination index (CI) of about 0.3 to about 0.9. In some embodiments, the targeted B-cell therapy or pharmaceutically acceptable salt thereof and additional therapy have a combination index (CI) of about 0.4 to about 0.9. In some embodiments, the targeted B-cell therapy or pharmaceutically acceptable salt thereof and additional therapy have a combination index (CI) of about 0.5 to about 0.9.

In some embodiments, the targeted B-cell therapy or pharmaceutically acceptable salt thereof and additional therapy have a combination index (CI) of about 0.01 to about 0.3. In some embodiments, the targeted B-cell therapy or pharmaceutically acceptable salt thereof and additional therapy have a combination index (CI) of about 0.05 to about 0.3. In some embodiments, the targeted B-cell therapy or pharmaceutically acceptable salt thereof and additional therapy have a combination index (CI) of about 0.8 to about 0.3.

### Targeted B-Cell Therapy Doses

In some embodiments, the targeted B cell therapy is ibrutinib or a pharmaceutically acceptable salt thereof.

The chemical name for ibrutinib is 1-[(3R)-3-[4-amino-3-(4-phenoxyphenyl)-1Hpyrazolo[3,4-d]pyrimidin-1-yl]-1-piperidinyl]-2-propen-1-one and has the following structure:

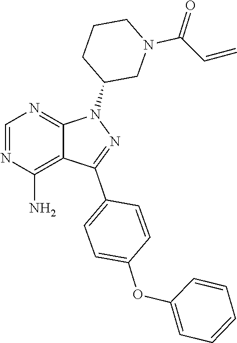 [SMILES: C=CC(=O)N1CCC[C@@H](n2nc(-c3ccc(Oc4ccccc4)cc3)c3c(N)ncnc32)C1]

In the United States, IMBRUVICA® (ibrutinib) capsules for oral administration are available in the following dosage strengths: 70 mg and 140 mg. Each capsule contains ibrutinib (active ingredient) and the following inactive ingredients: croscarmellose sodium, magnesium stearate, microcrystalline cellulose, sodium lauryl sulfate. The capsule shell contains gelatin, titanium dioxide, yellow iron oxide (70 mg capsule only), and black ink. Ibrutinib tablets for oral administration are available in the following dosage strengths: 140 mg, 280 mg, 420 mg, and 560 mg. Each tablet contains ibrutinib (active ingredient) and the following inactive ingredients: colloidal silicon dioxide, croscarmellose sodium, lactose monohydrate, magnesium stearate, microcrystalline cellulose, povidone, and sodium lauryl sulfate. The film coating for each tablet contains ferrosoferric oxide (140 mg, 280 mg, and 420 mg tablets), polyvinyl alcohol, polyethylene glycol, red iron oxide (280 mg and 560 mg tablets), talc, titanium dioxide, and yellow iron oxide (140 mg, 420 mg, and 560 mg tablets).

Ibrutinib (Ibruvica® Pharmacyclics; Abb Vie) is approved for:
Treatment of mantle cell lymphoma in adult patients who have received at least one prior therapy.
Treatment of chronic lymphocytic leukemia (CLL) or small lymphocytic lymphoma (SLL) [both including CLL and SLL with 17p deletion
Treatment of Waldenström's macroglobulinemia.
Marginal zone lymphoma (MZL) who require systemic therapy and have received at least one prior anti-CD20-based therapy
Chronic graft versus host disease (cGVHD) after failure of one or more lines of systemic therapy

### Dosage:

MCL and MZL: 560 mg taken orally once daily.
CLL/SLL, WM, and cGVHD: 420 mg taken orally once daily.

Dose should be taken orally with a glass of water. Do not open, break, or chew the capsules. Do not cut, crush, or chew the tablets.

In some embodiments, the targeted B cell therapy is acalabrutinib or a pharmaceutically acceptable salt thereof.

In the United States, acalabrutinib (Calquence® AstraZeneca Pharmaceuticals) is approved for:
Treatment of mantle cell lymphoma in adult patients who have received at least one prior therapy;
Treatment of chronic lymphocytic leukemia (CLL) or small lymphocytic lymphoma (SLL).

Recommended dose is 100 mg orally approximately every 12 hours; swallow whole with water and with or without food.

In some embodiments, the targeted B cell therapy is zanubrutinib or a pharmaceutically acceptable salt thereof.

In the United States, zanubrutinib (Brukinsa® Beigene, USA) is approved for:
Treatment of mantle cell lymphoma in adult patients who have received at least one prior therapy.

Recommended dose: 160 mg orally twice daily or 320 mg orally once daily; swallow whole with water and with or without food. Reduce dose in patients with severe hepatic impairment.

In some embodiments, the targeted B cell therapy is Tocilizumab or a pharmaceutically acceptable salt thereof.

In the United States, Tocilizumab (Actemra® Genentech) [IL-6r antibody] is approved for:
Treatment of Rheumatoid Arthritis (RA) in adult patients with moderately to severely active rheumatoid arthritis who have had an inadequate response to one or more Disease-Modifying Anti-Rheumatic Drugs (DMARDs);
Treatment of Giant Cell Arteritis (GCA) Adult patients with giant cell arteritis;
Treatment of Systemic Sclerosis-Associated Interstitial Lung Disease (SSc-ILD) Slowing the rate of decline in pulmonary function in adult patients with systemic sclerosis-associated interstitial lung disease (SSc-ILD);
Treatment of Polyarticular Juvenile Idiopathic Arthritis (PJIA) in patients 2 years of age and older with active polyarticular juvenile idiopathic arthritis;
Treatment of Systemic Juvenile Idiopathic Arthritis (SJIA) in patients 2 years of age and older with active systemic juvenile idiopathic arthritis; and
Treatment of Cytokine Release Syndrome (CRS) in adults and pediatric patients 2 years of age and older with chimeric antigen receptor (CAR) T cell-induced severe or life-threatening cytokine release syndrome.

Recommended Adult Intravenous (IV) dose for Rheumatoid Arthritis (RA) is 4 mg per kg every 4 weeks followed by an increase to 8 mg per kg every 4 weeks based on clinical response. Recommended Adult Subcutanenous (SC) dose for Rheumatoid Arthritis (RA) is 162 mg every other week followed by an increase to every week based on clinical response for patients weighing less than 100 kg, and 162 mg every week for patients weighing 100 kg or more.

Recommended Adult Subcutanenous (SC) dose for Giant Cell Arteritis (GCA) is 162 mg every other week in combination with a tapering course of glucocorticoids.

Recommended Intravenous (IV) dose for Polyarticular Juvenile Idiopathic Arthritis (PJIA) is 10 mg per kg every 4 weeks for patients weighing less than 30 kg, and 8 mg per kg every 4 weeks for patients weighing 30 kg or more.

Recommended Intravenous (IV) dose Systemic Juvenile Idiopathic Arthritis (SJIA) is 12 mg per kg every 2 weeks for patients weighing less than 30 kg, and 8 mg per kg every 2 weeks for patients weighing 30 kg or more.

Recommended Intravenous (IV) dose for Cytokine Release Syndrome (CRS) is 12 mg per kg for patients weighing less than 30 kg, and 8 mg per kg for patients weighing 30 kg or more, alone or in combination with corticosteroids.

In some embodiments, a CXCR4 inhibitor may be added as a third agent to the combination therapies. In some embodiments, the CXCR4 inhibitor is Mavorixafor.

Mavorixafor (X4P-001) is a CXCR4 antagonist, with molecular formula C₂₁H₂₇N₅; molecular weight 349.48 amu; and appearance as a white to pale yellow solid. Solubility: freely soluble in the pH range 3.0 to 8.0 (>100 mg/mL), sparingly soluble at pH 9.0 (10.7 mg/mL) and slightly soluble at pH 10.0 (2.0 mg/mL). Mavorixafor is only slightly soluble in water. Melting point: 108.9° C.

The chemical structure of mavorixafor is depicted below.

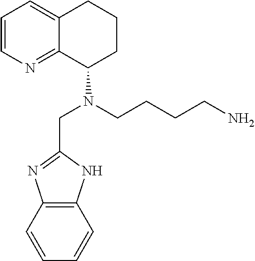 [SMILES: NCCCCN(Cc1nc2ccccc2[nH]1)[C@H]1CCCc2cccnc21]

In certain embodiments, a pharmaceutical composition containing mavorixafor or a pharmaceutically acceptable salt thereof is administered orally in an amount from about 200 mg to about 1200 mg daily. In certain embodiments, the dosage composition may be provided twice a day in divided dosage, approximately 12 hours apart. In other embodiments, the dosage composition may be provided once daily. The terminal half-life of mavorixafor has been generally determined to be between about 12 to about 24 hours, or approximately 14.5 hrs. Dosage for oral administration may be from about 100 mg to about 1200 mg once or twice per day. In certain embodiments, the dosage of mavorixafor or a pharmaceutically acceptable salt thereof useful in the invention is from about 200 mg to about 600 mg daily. In other embodiments, the dosage of mavorixafor or a pharmaceutically acceptable salt thereof useful in the invention may range from about 400 mg to about 800 mg, from about 600 mg to about 1000 mg or from about 800 mg to about 1200 mg daily. In certain embodiments, the invention comprises administration of an amount of mavorixafor or a pharmaceutically acceptable salt thereof of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, or about 1600 mg.

In some embodiments, a provided method comprises administering to the patient a pharmaceutically acceptable composition comprising mavorixafor or a pharmaceutically acceptable salt thereof wherein the composition is formulated for oral administration. In certain embodiments, the composition is formulated for oral administration in the form of a tablet or a capsule. In some embodiments, the composition comprising mavorixafor or a pharmaceutically acceptable salt thereof is formulated for oral administration in the form of a capsule.

In certain embodiments, a provided method comprises administering to the patient one or more unit doses, such as capsules, comprising 100-1200 mg mavorixafor or a pharmaceutically acceptable salt thereof as an active ingredient; and one or more pharmaceutically acceptable excipients.

The term “patient,” as used herein, means an animal, preferably a mammal, and most preferably a human.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As used herein, “about” means that the stated value or range may vary by up to 10% from the stated value or range. For example, “about” 5.0 means 5.0±0.5, and “about 5.0-10.0” means 4.5-10.5.

The term “pharmaceutically acceptable carrier, adjuvant, or vehicle” refers to a non-toxic carrier, adjuvant, or vehicle that does not destroy the pharmacological activity of the compound with which itis formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a patient, is capable of providing, either directly or indirectly, a compound of this invention.

Compositions of the present invention may be administered orally, parenterally, by inhalation spray, topically (as by powders, ointments, or drops), rectally, nasally, buccally, intravaginally, intracisternally, or via an implanted reservoir. The term “parenteral” as used herein includes subcutaneous, intravenous, intramuscular, intra-articular, intra-synovial, intrasternal, intrathecal, intrahepatic, intralesional, and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

The compounds and compositions, according to the method of the present invention, may be administered using any amount and any route of administration effective for treating a cancer, such as those disclosed herein. The exact amount required will vary from subject to subject, depending on the species, age, and general condition of the subject, the severity of the cancer, the particular agent, its mode of administration, and the like. Compounds of the invention are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the cancer being treated and the severity of the cancer; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus, the kit of the invention includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

The contents of each document cited in the specification are herein incorporated by reference in their entireties.

### EXEMPLIFICATION

### Example 1: Mavorixafor Disrupts the Crosstalk Between Waldenström's Macroglobulinemia Cells and the Bone Marrow Stromal Cells and Enhances their Sensitivity to B-Cell-Targeted Therapies

Waldenström's macroglobulinemia (WM) is a rare indolent B-cell lymphoma characterized by excess accumulation of malignant lymphoplasmacytic cells in the bone marrow (BM) and hypersecretion of monoclonal immunoglobulin M (IgM) by WM cells. Infiltration of BM by malignant cells and crosstalk with the BM microenvironment are thought to contribute to pathogenesis and resistance to therapy in WM. Here, we aimed to examine the crosstalk between WM cells and bone marrow stromal cells (BMSCs) in an in vitro coculture model.

Additionally, we sought to determine the effects of mavorixafor, an orally available CXCR4 antagonist, alone or in combination with B-cell-targeted drugs, including Bruton's tyrosine kinase antagonists or a B-cell lymphoma 2 inhibitor on WM cells in coculture with BMSCs. Our results demonstrate that in cocultures of WM cells with BMSCs, BMSC-derived interleukin-6 (IL-6) upregulated IgM secretion and increased CXCR4 cell surface expression in WM cells via IL-6R-janus kinase-signal transducer and activator of transcription-3 signaling, and increased WM cells adhesion to BMSCs. In cocultures, BMSCs led to reduced apoptosis of WM cells treated with the tested B-cell-targeted inhibitors, suggesting BMSCs conferred drug resistance in WM cells. Blocking the CXCR4/CXLC12 axis with mavorixafor alone or in combination with tested B-cell-targeted inhibitors resulted in disruption of WM cell migration and adhesion to BMSCs, enhanced antitumor activity of B-cell-targeted inhibitors, overcame BMSC-induced drug resistance, and reduced BMSC-induced IgM hypersecretion. These results provide support for the potential use of mavorixafor alone or in combination with B-cell-targeted drugs for the treatment of WM and possibly for other malignancies.

Waldenström's macroglobulinemia (WM) is an indolent B-cell lymphoma characterized by accumulation of malignant lymphoplasmacytic cells in the bone marrow (BM) (1). High levels of monoclonal immunoglobulin M (IgM) are secreted by WM cells, resulting in anemia, blood hyperviscosity syndrome, visual impairments, and neurological symptoms (2). Consequently, lowering serum IgM is a key end point in WM therapy and a common parameter to assess the success of any WM treatment.

Over the last decade, significant progress has been made in understanding the genetics underlying the pathogenesis of WM. Somatic mutations in clonal populations of cells lead to WM. The somatic L265P mutation in MYD88 innate immune signal transduction adaptor (MYD88) gene can be found in >90% of patients with WM (3,4). The MYD88 gene encodes a protein that is involved in signaling pathways, including activation of nuclear factor-kB upon stimulation of toll-like receptors (TLRs). Additionally, MYD88 anchors with phosphorylated Bruton tyrosine kinase (BTK), which itself is part of many signaling pathways, including toll-like, chemokine and B-cell receptors (5). MYD88^{L265P} is thought to be an activating mutation that increases binding to BTK, promoting cell survival and proliferation (3).

A second, more diverse category of mutation in WM, detected in approximately 30% of patients, can be found in the gene encoding C-X-C chemokine receptor 4 (CXCR4) (6-8). The G protein-coupled receptor CXCR4 binds its natural ligand C-X-C chemokine ligand 12 (CXCL12), which is produced by the perivascular cells of the bone marrow stroma. Upon binding to CXCR4, CXCL12 induces downstream signaling activation of phosphoinositide 3-kinase (PI3K), which controls lymphocyte trafficking, chemotaxis, and cell survival (9,10). In WM, CXCR4 mutation generally occurs in the C terminal, intracellular domain of the protein-a region involved in signal transduction. Most CXCR4 C-terminal mutations found in WM cause hyperactivation of the receptor and its downstream signaling pathways, resulting in decreased internalization of the receptor and increased chemotaxis (7,11,12). Patients with MYD88^{L265P} CXCR4^{Mut} WM typically present with higher serum IgM levels and greater BM involvement compared with those with MYD88^{L265P} mutation alone (3,13).

Until recently, the typical therapeutic regimen for patients with WM consisted of chemotherapy, usually combined with targeted therapy such as rituximab (14,15). In 2015, the BTK inhibitor ibrutinib was the first drug to be approved by the US Food and Drug Administration specifically for WM, followed by the next-generation BTK inhibitor zanubrutinib. Many more compounds with similar properties are currently being tested in clinical trials (14,15). Despite these advancements, it became clear that mutational status is a crucial determinant in the therapeutic management of WM (16-19). Patients harboring MYD88^{L265P} and CXCR4 mutations have a lower response to BTK inhibitors and lower progression-free survival rates vs patients with the MYD88^{L265P} mutation alone (16-19). This observation led to a recent clinical trial (NCT04274738) designed to test the effects of combination therapy with ibrutinib and mavorixafor. Mavorixafor is a noncyclam, orally available CXCR4 antagonist previously shown to effectively block the CXCL12-induced signaling pathway in acute lymphocytic leukemia (ALL) cells (20). Moreover, mavorixafor is currently under investigation in clinical trials for patients with Warts, Hypogammaglobulinemia, Infections, and Myelokathexis (WHIM) syndrome (NCT03995108) and severe congenital neutropenia (SCN)/chronic idiopathic neutropenia (CN) (NCT04154488).

The interactions of malignant lymphoplasmacytic cells with the BM microenvironment (via cell-cell adhesion or secreted factors) are thought to contribute to the pathogenesis of disease and cause drug resistance in WM cells (21-24). Higher levels of cytokines and chemokines (primarily CXCL12) are detected in the BM of patients with WM compared with healthy controls (22). The elevated CXCL12 may be responsible for increased homing of lymphoplasmacytic cells to the BM (24). Additionally, higher levels of interleukin 6 (IL-6) are found in BM and sera of patients with WM (22,25). To note, IL-6 directly contributes to IgM production in in vitro and in vivo models of WM, and in both models, treatment with an IL-6 receptor (IL-6R) antibody reduces IgM levels (26,27). IL-6 signaling links to signal transducer and activator of transcription 3 (STAT3) signaling, a pathway disrupted in many cancers. Preclinical work in WM demonstrated upregulation of IL-6/STAT3 signaling components, and a STAT3 inhibitor showed in vitro efficacy in WM cell lines (22,28,29). The BM microenvironment-mediated tumor progression and drug resistance involving CXCR4/CXCL12 and IL-6/STAT3 axis are also well recognized in various malignancies (e.g., ALL, chronic myelocytic leukemia [CML], chronic lymphocytic leukemia, multiple myeloma [MM], and diffuse large B-cell lymphoma [DLBCL]) (20,30-33).

Interestingly, recent data suggest that patients with MYD88 mutation but wild-type (WT) CXCR4 still exhibit CXCR4 dysregulation. A study comparing CXCR4 expression in samples from patients with MYD88^{L265P}CXCR4^{WT} and MYD88^{L265P}CXCR4^{WHIM} WM showed almost equally high increases in CXCR4 expression compared with healthy controls (34,35). These findings suggest that dysregulation of CXCR4, CXCL12, and subsequent downstream signaling events could contribute to disease pathogenesis, even in MYD88^{L265P}CXCR4^{WT} WM.

In the current study, we aimed to investigate the crosstalk between WM cell lines harboring MYD88^{L265P}CXCR4^{WT} and bone marrow stromal cells in an in vitro coculture model. We also sought to evaluate the effect of mavorixafor with and without B-cell-targeted drugs, including BTK inhibitors or a B-cell lymphoma 2 inhibitor, on WM cells carrying only the MYD88^{L2659} mutation in coculture with BMSCs.

### Material and Methods

### Drugs and Reagents

The BTK inhibitor evobrutinib (#S8777) was provided by Selleck chemicals. The BTK inhibitors ibrutinib (#HY-10997/CS), zanubrutinib (#HY-101474A), pirtobrutinib (#HY-131328), nemtabrutinib (#HY-112215), B-cell lymphoma 2 (BCL2) inhibitor venetoclax (#HY-15531), STAT3 inhibitor BP-1-102 (#HY-100493), pan-janus kinase (JAK) inhibitor PF-06263276 (#HY-101024), and IL-6R antibody tocilizumab (#HY-P9917) were purchased from MedChemExpress. Mavorixafor was synthesized by ChemPartner. Human CXCL12 (#300-28A) and IL-6 (#200-06) were purchased from PeproTech.

### Cell Lines

MWCL-1 cells were provided by Dr Stephen M. Ansell (MAYO file number 2021-121; 200 First Street SW, Rochester, Minnesota) and the bone marrow stroma cell (BMSC) lines HS-27A and HS-5 were obtained from ATCC. All cell lines were cultured in RPMI-1640 medium (Fisher Scientific, #32404-014) containing 10% fetal bovine serum (FBS) (Sigma-Aldrich, #F7524 or Takara Bio, #631105), supplemented with 100 U/mL of Penicillin-Streptomycin (Gibco™, Thermo Fisher Scientific, #11548876) at 37° C. and 5% CO₂.

### Coculture Experiments

BMSCs were cultured in 96-, 48-, or 24-well plates until 90% confluence. MWCL-1 cells (density≈2×10⁵ cells/mL) were pretreated with indicated concentration of mavorixafor together with indicated concentrations of B-cell-targeted inhibitors in medium containing 4% FBS for 1 hour and transferred to the BMSC monolayer. Cells were coincubated for 48 or 72 hours followed by measurement of cell viability, apoptosis, IgM, and IL-6 release.

### Cell Viability Assay

Cellular viability (as measured using metabolic activity) was determined using the CellTiter-Glo® assay (Promega, #G7570) according to the manufacturer's instructions.

### Enzyme-Linked Immunosorbent Assays

The IgM levels were quantitated using a human IgM Enzyme-linked immunosorbent assay (ELISA) kit (Abcam, #ab214568) according to the manufacturer's instructions. IL-6 levels were quantitated using an IL-6 ELISA MAX™ kit (BioLegend, #430515) per manufacturer's recommendations. For all ELISA kits, plates were developed with 3,3′,5,5′-tetramethylbenzidine (TMB) development solution or a biotinylated antihuman IL-6 detection antibody/avidin horseradish peroxidase (HRP) solution. The reaction was stopped with the stop solution, and absorbance was read at 450 nm with a microplate reader (Synergy™ HT, BioTek Instruments).

### Calcium Mobilization Assay

MWCL-1 cells (2×10⁵ cells/well) were seeded in transparent bottom, black 96-well plates coated with poly-L-lysine (BioCoat®, Corning) and serum-starved (medium with 1% FBS) for 24 hours. Medium was removed and cells were loaded with 100 μL of fluo-4 AM (3 μM, Invitrogen, #F14201) dye solution for 45 minutes at 37° C. Subsequently, 100 μL of assay buffer alone or assay buffer with compound dilutions was added, and the plates were equilibrated in the plate reader for an additional 20 minutes at 37° C. The CXCL12 was injected with simultaneous measurement of fluorescent signal (FlexStation® 3 Multi-Mode Microplate Reader, Molecular Devices). Raw traces were analyzed in SoftMax®Pro 7 Software (Molecular Devices). The arbitrary units were calculated as the difference between maximal and minimal signal after treatment injection, normalized to the baseline signal before injection.

### Transwell Migration Assay

Cell migration toward CXCL12 gradient or BMSC monolayer was determined using the Transwell migration assay. MWCL-1 cells were stained with 500 nM Calcein AM (Invitrogen, #C1430) and preincubated with mavorixafor for 15 minutes before transfer (5×10⁵ cells) to an upper well of a 5.0 μM pore size Transwell® (Corning, #3421). The lower chamber contained either CXCL12 (10 nM) in medium supplemented with 1% FBS or a monolayer of HS-27A BMSCs seeded 72 hours prior and starved for 48 hours with 4% FBS medium. Wells without CXCL12 were treated with 10 μM LIT-927 (Selleck Chemicals, #S8813), a neutraligand of CXCL12, to serve as background control. After 4 hours of incubation, cells that migrated to the lower chambers were collected and resuspended in Dulbecco's phosphate-buffered saline (DPBS) containing Precision Count Beads™ (BioLegend, #424902). Migrated cells and counting beads were counted by flow cytometry

### Immunoblotting

MWCL-1 mono- or cocultures were treated with mavorixafor and/or ibrutinib for 24 hours. Whole cells were lysed by radioimmunoprecipitation assay (RIPA) lysis buffer (Sigma-Aldrich, #R0278) with protease inhibitor cocktail (Roche Custom Biotech, #11697498001). Lysates were separated by sodium dodecyl sulfate polyacrylamide gel-electrophoresis (SDS-PAGE) electrophoresis and transferred to Trans-Blot® Turbo™ Mini PVDF Transfer Packs (Bio-Rad). Membranes were incubated with primary antibodies: anti-poly adenosine diphosphate (ADP)-ribose polymerase (PARP) (1:1000, Cell Signaling, #9542); Anti-Caspase 3 (1:1000, Cell Signaling, #9662) and antitubulin (1:5000, R&D, #MAB9344). Secondary HRP-conjugated antibodies (Abcam) were used at 1:10.000 dilution. Membranes were developed with enhanced chemiluminescence reagent (Amersham™ ECL Prime Western Blotting Detection Reagent, GE Healthcare) on LAS4000 gel documentation system.

### Flow Cytometry

Flow experiments were performed with the CytoFLEX S Flow Cytometer (Beckman Coulter). Samples were analyzed using FCS Express Software (De Novo Software).

### Adhesion Assay

MWCL-1 cells were labeled with 1 μM Calcein AM. After 10 minutes at 37° C., cells were washed, resuspended in medium containing 1% FBS, and treated with compounds for 30 minutes (2.25× 10⁵ cells/mL). MWCL-1 cells were transferred to the BMSC monolayer. After 4 hours, nonadherent cells were removed by gently washing with phosphate-buffered saline (PBS). Remaining cells were harvested, resuspended in flow buffer (Hanks balanced salt solution+20 mM HEPES+0.5% body surface area) and analyzed by flow cytometry.

### Apoptosis Assay

Cells were washed with PBS, resuspended in Annexin V binding buffer (BioLegend, #422201), and stained with Alexa Fluor® 647 Annexin V (BioLegend, #640943), propidium iodide (BD Pharmingen, #51-66211E), and CD45 antibody (BioLegend, #368502) for 15 minutes at room temperature and analyzed by flow cytometry.

### CXCR4 Expression

MWCL-1 cells were pretreated with tocilizumab, BP-1-102, or PF-06263276 for 20 minutes and stimulated with IL-6 for 24 hours. Cells were stained with CXCR4 antibody (BD Pharmigen, #555976) and measured by flow cytometry.

### Phosphoflow

MWCL-1 cells were seeded in starvation medium in 96-well plate overnight. After stimulation with IL-6, the cells were fixed and permeabilized using BD Phosflow™ Fix Buffer I (BDBiosciences, #557870) and BD Phosflow™ Perm Buffer III (BDBiosciences, #558050) according to the manufacturer's instructions. Cells were then stained with Brilliant Violet 421 Mouse Anti-Phospho STAT3 (pS727, BDBioscience, #565416), Pacific Blue™ Mouse Anti-Phospho-STAT3 (pY705, BDBioscience, #560312), Alexa Fluor® 647 Mouse Anti-STAT5 (pY694, BDBioscience, #612599), AF488 Mouse Anti-Phospho-p38 MAPK (Thr180/Tyr182, Cell Signaling Technology, #4551), Mouse Anti-Phospho-JNK (Thr183/Tyr185, Cell Signaling, #9275S), and PE Mouse Anti-Phospho-IkB (Ser32/Ser36, Thermo Fisher, #12-9035-42) for 1 hour at room temperature in darkness. Cells were then washed 2 times in BD Pharmingen™ Stain Buffer (BD Pharmingen, #554656), resuspended in flow buffer, and measured by flow cytometry.

### Statistical and Bioinformatic Analysis

For each experiment, independent biological replicates were performed; their numbers are indicated in the figure legends. Significance of differences between 2 independent groups was calculated using Student two-tailed t test; significance of differences between multiple groups was determined by 2-way analysis of variance (ANOVA) followed by Bonferroni post hoc test. Analyses were performed using GraphPad Prism 9 software (GraphPad Software). The combination index (CI) was calculated using Chou-Talalay method (36). A CI of 1 indicates an additive effect, a CI<1 a synergistic effect, and a CI>1 an antagonistic effect. To compare the expression of CXCR4 between B-cells derived from patients with WM and healthy donors, the data sets GSE171739 and GSE9656 were used. Statistical significance was assessed using the ImFit function of the R package Linear Models for Microarray (Limma) as previously described (37). False discovery rates <0.1 were considered significant.

### Results

### BMSC-Derived IL-6 Causes IgM Hypersecretion by WM Cells Via IL-6R-JAK-STAT3

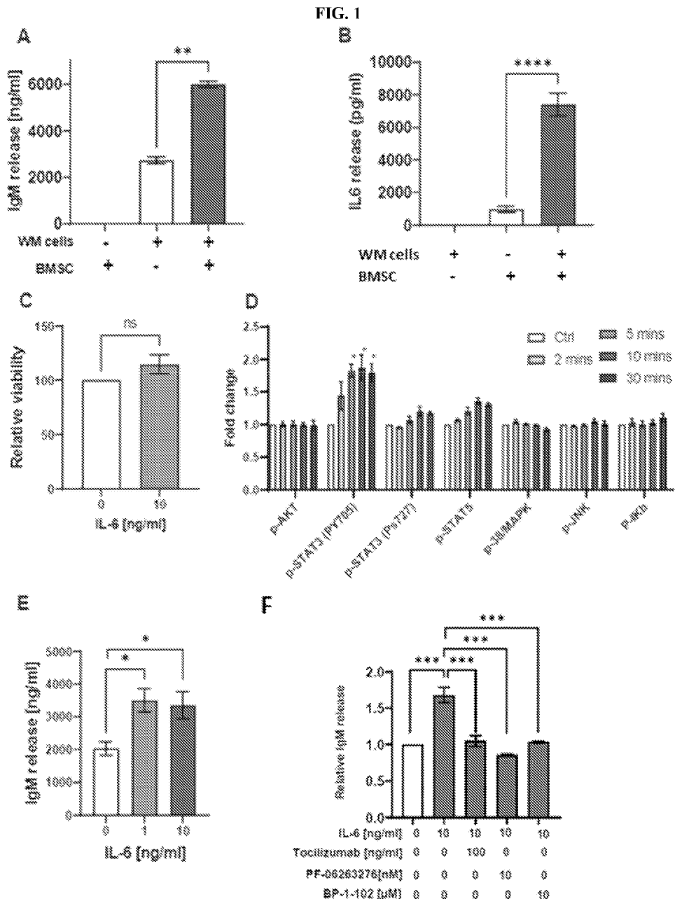

Since WM is characterized by BM infiltration with malignant lymphoplasmacytic cells with increased synthesis of IgM (1,3), we tested whether BMSCs affected IgM secretion by MWCL-1 WM cells. HS-27A BMSCs were cocultured with MWCL-1 cells for 72 hours, and IgM levels were measured in cell culture supernatants. MWCL-1 cells, but not HS-27A BMSCs, secreted IgM into the supernatant, and coculture with HS-27A BMSCs led to an increase in IgM secretion (FIG. 1A).

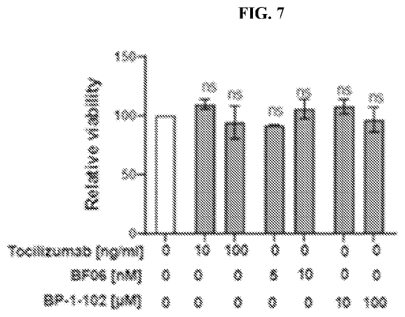

Since IL-6 signaling was previously linked to IgM production and secretion in WM cells (25,38), the cocultures were tested for the presence of the cytokine. HS-27A BMSCs, but not MWCL-1 cells, secreted IL-6, and coculture of these cells with WM cells increased IL-6 levels (FIG. 1B). In an analysis of its effects on WM cells, IL-6 showed minimal impact on the viability of MWCL-1 cells (FIG. 1C). However, IL-6 specifically caused phosphorylation of STAT3, and enhanced IgM secretion in MWCL-1 cells, whereas other pathways were much less affected (FIG. 1D, E). IL-6-mediated IgM secretion in WM cells was inhibited by treatment with the IL-6R antibody tocilizumab, pan-JAK inhibitor PF-06263276, or STAT3 inhibitor BP-1-102 (FIG. 1F). Of note, viability of MWCL-1 cells was not affected by these inhibitors (FIG. 7). These findings suggest that the effects of these inhibitors on IgM secretion in MWCL-1 cells were a direct result of activation of the intracellular signaling, independent of reduced cell viability.

These data suggest that BMSC-derived IL-6 upregulates IgM secretion in WM cells via the IL-6R-JAK-STAT3 pathway.

### BMSC-Derived IL-6 Increases CXCR4 Cell Surface Expression in WM Cells Via IL-6R-JAK-STAT3 Signaling and Enhances WM Cell Adhesion to BMSCs

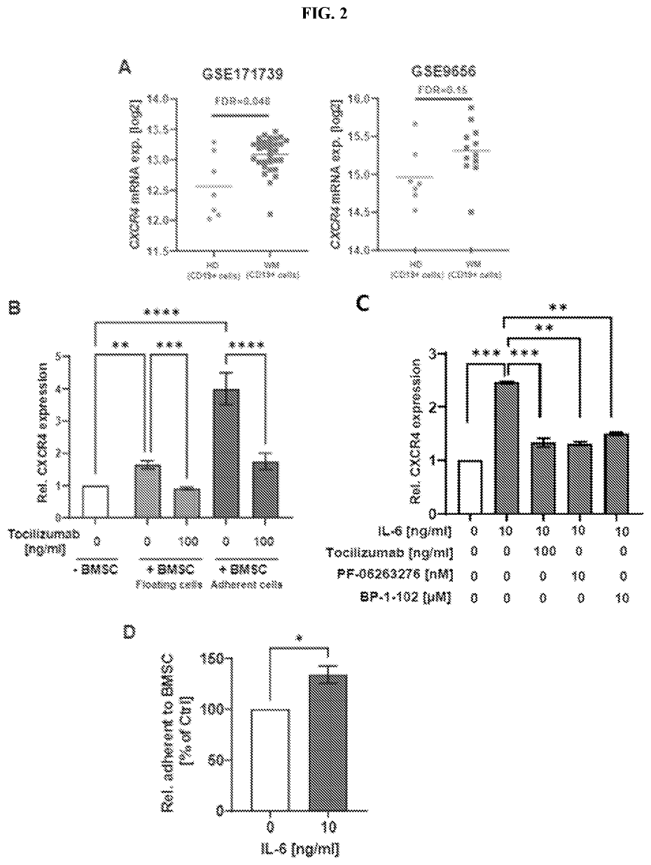

The CXCR4/CXCL12 axis plays an essential role in the homing of malignant cells to the protective niche of the BM (24,39,40), and CXCR4/CXCL12 axis expression is upregulated in malignant cells and BM of patients with WM (24,34,35). To confirm and extend these findings, expression of CXCR4 in several publicly available gene expression data sets was analyzed (GSE171739 and GSE9656). CXCR4 expression was significantly upregulated in B cells derived from the BM of patients with WM compared with peripheral B cells from healthy donors (FIG. 2A), suggesting that the BM microenvironment may have a direct impact on CXCR4 expression in WM cells.

Notably, whereas MWCL-1 cells alone expressed medium CXCR4 levels (MFI 1500-2000), coculture with HS-27A BMSCs significantly increased CXCR4 surface expression in MWCL-1 cells, with a more pronounced effect in the adherent cell population (MFI 8500-10000) (FIG. 2B). This increase was inhibited with an IL-6R antibody, suggesting dependence on IL-6 signaling. CXCR4 surface expression was also increased in MWCL-1 cells stimulated with soluble IL-6 and this enhance can be blocked by tocilizumab, the pan-JAK inhibitor BP-1-102, and the STAT-3 inhibitor PF-06263276 (FIG. 2C).

As overexpression of CXCR4 in WM cells has been shown to increase cellular adhesion to BMSCs (41), and IL-6 enhances CXCR4 surface expression in WM cells, we explored whether increased IL-6 exposure would increase WM cell adhesion to BMSCs. Pretreatment of MWCL-1 cells with soluble IL-6 for 24 hours significantly promoted their adhesion to BMSCs (FIG. 2D). These findings indicate that BMSC-derived IL-6 enhances CXCR4 surface expression in WM cells via the IL-6R-JAK-STAT3 axis, increasing their adhesion to BMSCs.

### BMSC-Induced Resistance of WM Cells to B-Cell-Targeted Therapies

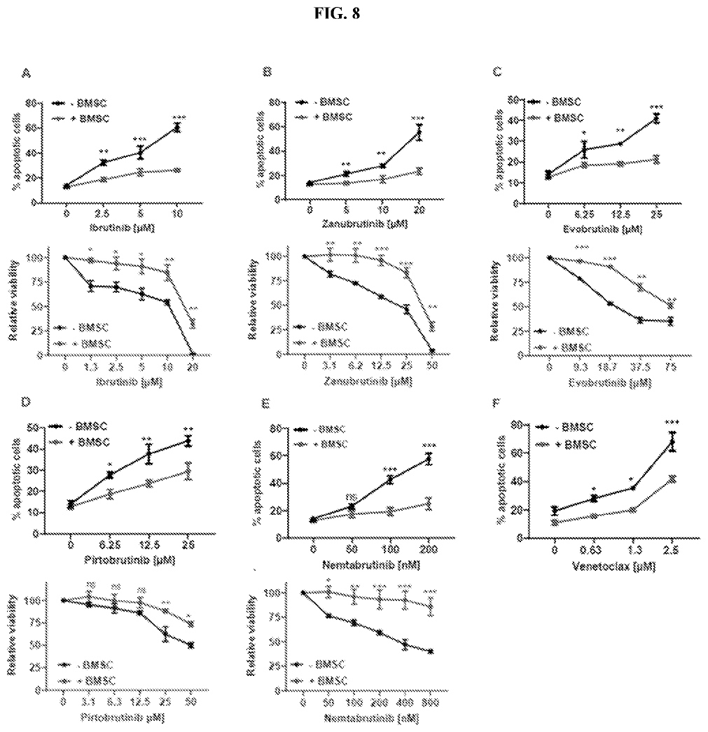

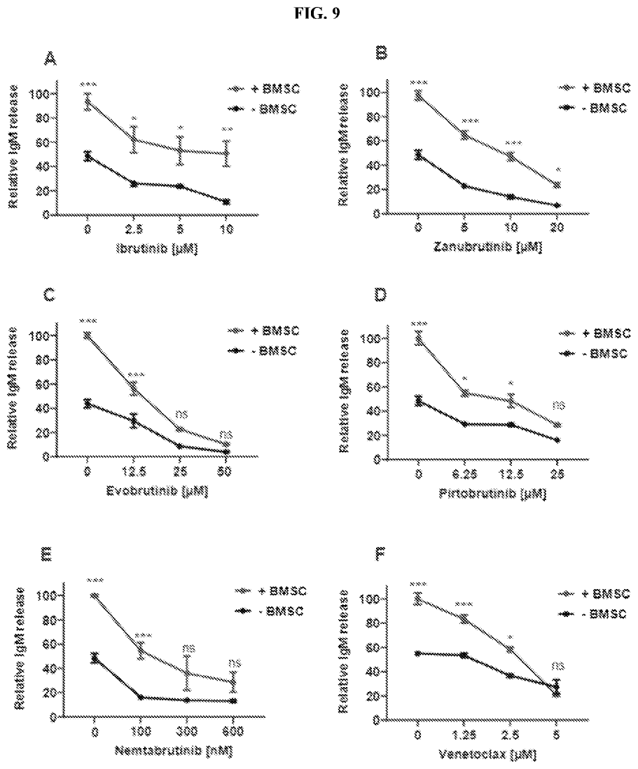

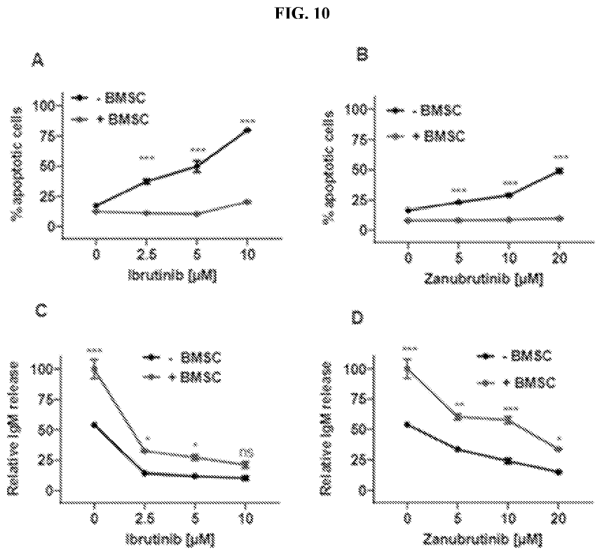

The crosstalk between malignant cells and the BM microenvironment was previously reported to cause drug resistance in various malignant cells (21,30,32,39,40,42). We explored whether BMSCs also conferred resistance of WM cells to different B-cell-targeted drugs. MWCL-1 cells were treated with various B-cell-targeted inhibitors both in mono- and in coculture with BMSCs, and apoptosis, viability, and IgM secretion were measured. B-cell-targeted drugs in current use for treatment of patients with WM (BTK inhibitors ibrutinib and zanubrutinib) (14,15) or in/under review ongoing clinical trial for WM (BTK inhibitors evobrutinib, pirtobrutinib, nemtabrutinib; BCL-2 inhibitor venetoclax) (NCT03740529, NCT03162536, NCT02677324) were included in our study. All B-cell-targeted inhibitors tested led to a dose-dependent increase in apoptosis and decreased viability of MWCL-1 cells (FIG. 8A-F). Coculture with HS-27A BMSCs significantly reduced apoptosis and preserved the viability of MWCL-1 cells treated with B-cell-targeted inhibitors (FIG. 8A-F). Additionally, all tested B-cell-targeted drugs decreased IgM secretion by MWCL-1 cells in a dose-dependent manner, whereas HS-27A BMSCs coculture significantly reduced the drug-induced reduction of IgM secretion (FIG. 9A-F). Similarly, HS-5 BMSCs also reduced the sensitivity of MWCL-1 cells to B-cell-targeted drugs (ibrutinib and zanubrutinib) (FIG. 10A-D).

Our data indicate that BMSCs confer resistance of WM cells to all tested B-cell-targeted inhibitors.

### Mavorixafor Causes Disruption of WM Cell Migration and Adhesion to BMSCs

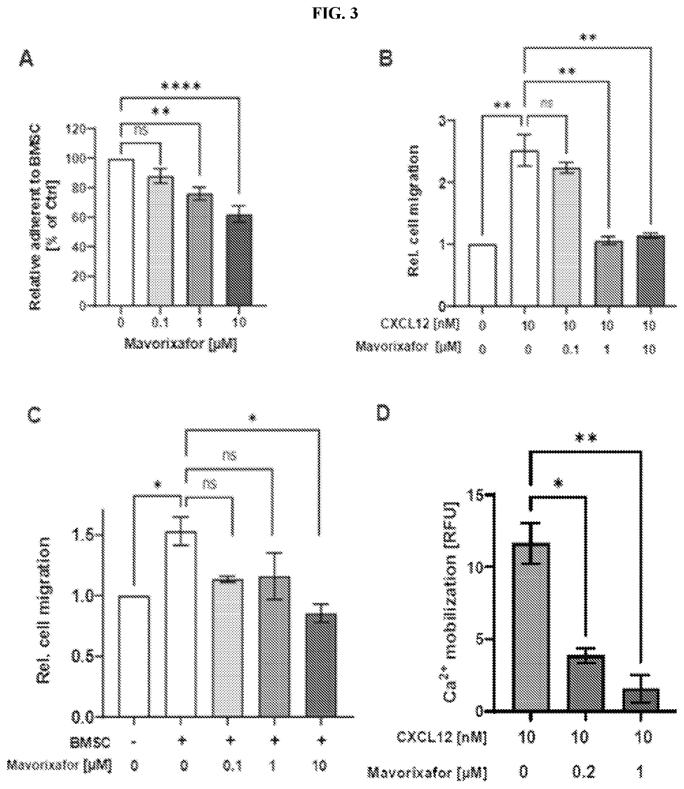

Because BMSC coculture induced upregulation of CXCR4 surface expression in WM cells and conferred resistance to different B-cell-targeted inhibitors, we sought to test whether these 2 observations were connected. We speculated that pharmacological blockage of the CXCR4/CXCL12 axis could disrupt the communication between WM cells and BMSCs and enhance sensitivity to B-cell-targeted inhibitors. MWCL-1 cells were preincubated with mavorixafor, an orally available CXCR4 antagonist that is currently being evaluated in clinical trials for patients with WHIM syndrome (NCT03995108), WM (NCT04274738) and SCN/CIN (NCT04154488), followed by the assessment of the percentage of MWCL-1 cells adhering to BMSCs. A dose-dependent decrease in adhesion of WM cells to BMSCs was observed after pretreatment with mavorixafor (FIG. 3A). The tested concentrations and durations (4 hours) were not sufficient to induce cytotoxicity in the MWCL-1 cells (data not shown). The effects of mavorixafor on the migration of WM cells in response to exogenous CXCL12, as well as to CXCL12 constitutively secreted by HS-27A BMSCs, were also assessed. Pretreatment of MWCL-1 cells with mavorixafor significantly inhibited their migration toward a CXCL12 gradient or BMSC-secreted CXCL12 (˜700-1000 μg/mL) (FIG. 3B, C). Lastly, since CXCL12-induced Ca²⁺ mobilization was associated with cell migration (43), we tested the effect of mavorixafor on CXCL12-induced Ca²⁺ influx in MWCL-1 cells. Mavorixafor blocked CXCL12-mediated Ca²⁺ mobilization in MWCL-1 cells in a dose-dependent manner (FIG. 3D). These findings indicate that mavorixafor disrupts the migration and adhesion of WM cells to BMSCs.

### Mavorixafor Enhances Antitumor Activity of B-Cell-Targeted Inhibitors in WM Cells and Overcomes BMSC-Induced Drug Resistance

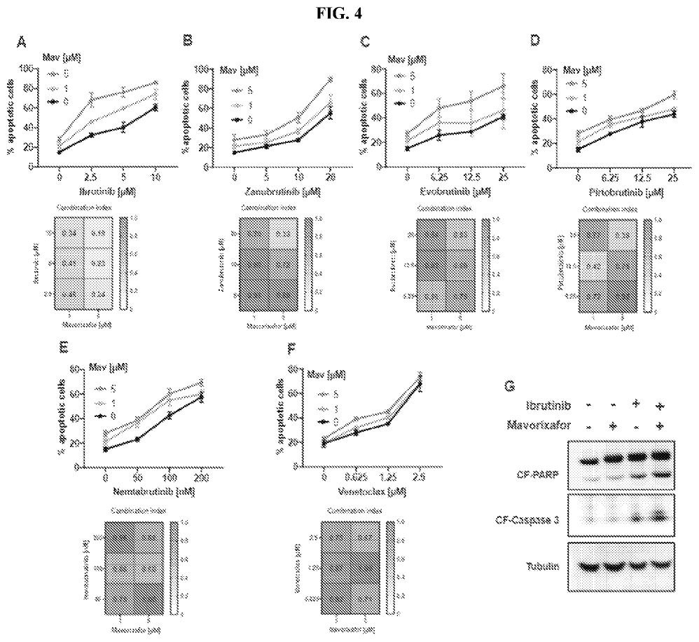

Next, we asked whether the disruption of WM cell adhesion to BMSCs by mavorixafor restores sensitivity to B-cell-targeted inhibitors. MWCL-1 cells were pretreated with mavorixafor alone or in combination with different B-cell-targeted inhibitors and cultured alone or together with HS-27A BMSCs. Apoptosis and viability were measured after 48 to 72 hours. Mavorixafor alone caused a minor increase in apoptosis of MWCL-1 cells (FIG. 4A-F). The combination of mavorixafor with B-cell-targeted inhibitors led to a further increase in apoptosis of MWCL-1 cells in monoculture (without stromal cells). Chou and Talalay analysis confirmed synergistic activity in most of the dose combinations (CI<1.0, FIG. 4A-F). These data suggest that mavorixafor enhances antitumor activity of all tested therapeutic agents. To further confirm this finding, MWCL-1 cells were cotreated with mavorixafor and ibrutinib, and the expression of proapoptotic proteins was analyzed by immunoblotting. Mavorixafor alone had minor effects on the cleavage of caspase-3 and PARP; however, combination with ibrutinib resulted in an increase in proteolytic cleavage of caspase-3 and PARP in MWCL-1 cells (FIG. 4G).

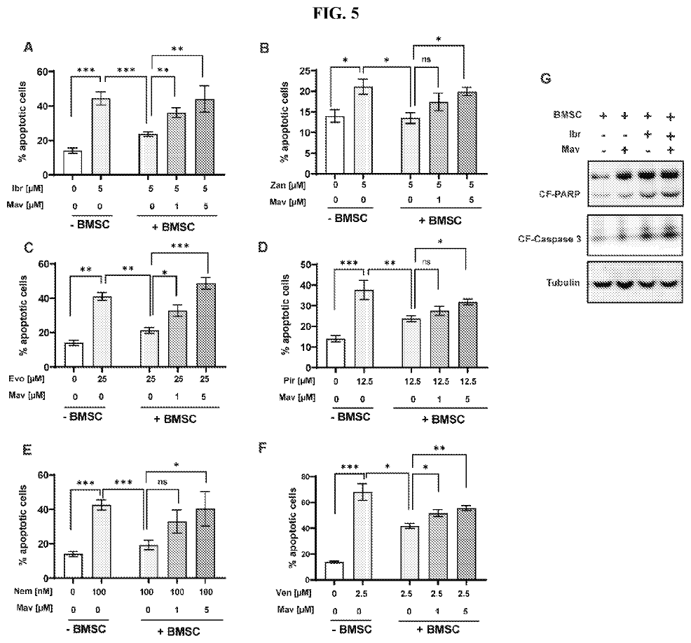

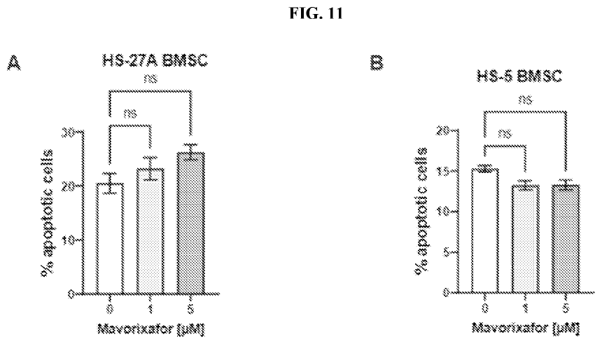

MWCL-1 cells, in the presence of BMSCs, showed resistance to apoptosis induced by all B-cell-targeted inhibitors tested. The addition of mavorixafor restored the sensitivity of MWCL-1 cells to all tested drugs (FIG. 5A-F). Of note, mavorixafor alone, at tested concentrations, had much weaker effects on apoptosis of BMSCs in a WM cell-BMSC cocultured model (FIG. 11A-B). To further explore the molecular mechanisms by which mavorixafor restored sensitivity to ibrutinib in the context of the BM microenvironment, we assessed the expression of proapoptotic proteins in MWCL-1 cells cultured with HS-27A BMSCs. As in monoculture, the combination of the 2 drugs led to a further enhanced proteolytic cleavage of caspase-3 and PARP in MWCL-1 BMSC cocultures (FIG. 5G).

Our data suggest that mavorixafor synergizes with B-cell-targeted inhibitors to enhance cytotoxicity in WM cells by inducing proapoptotic pathways and overcomes BMSC-mediated resistance to these drugs.

Mavorixafor as a Single Agent or in Combination with B-Cell-Targeted Therapies Inhibits BMSC-Induced IgM Hypersecretion

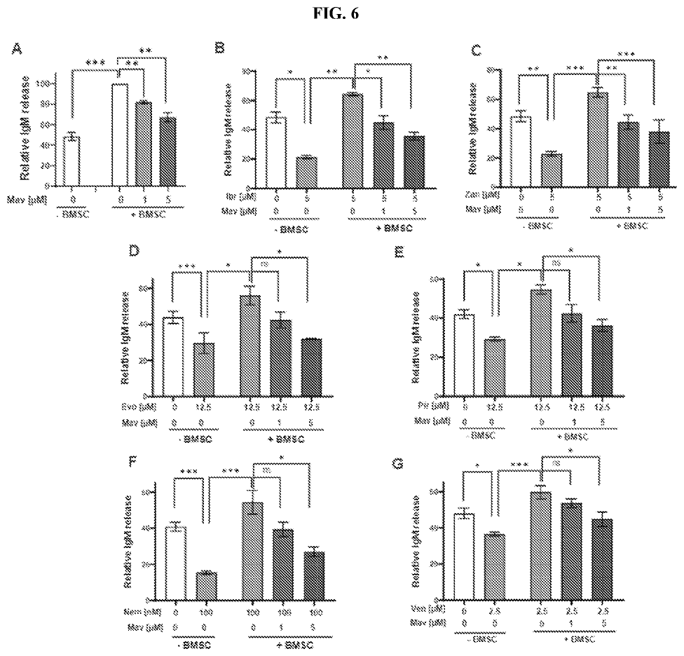

Since lowering of serum IgM is a primary outcome of WM therapy, we next asked whether mavorixafor as a single agent or in combination with B-cell-targeted agents could disrupt BMSC-mediated IgM hypersecretion by WM cells. MWCL-1 cells were preincubated with mavorixafor, B-cell-targeted inhibitors, or both, and cultured with or without HS-27A BMSCs, followed by supernatant IgM measurements after 48 or 72 hours. Mavorixafor alone inhibited BMSC-induced IgM hypersecretion in MWCL-1 cells in a dose-dependent fashion, while combination of mavorixafor with all tested B-cell-targeted inhibitors led to a further decreased BMSC-induced IgM hypersecretion in MWCL-1 cells (FIG. 6A-G).

Taken together, our data indicate that mavorixafor inhibits BMSC-mediated IgM hypersecretion and synergizes with all tested B-cell-targeted inhibitors to more effectively reduce IgM hypersecretion caused by BMSCs.

### DISCUSSION

Despite significant advances in our understanding of the pathophysiology and treatment of WM, it remains an incurable disease. Current therapeutic options for WM rely on the use of drugs targeting malignant cells directly, including BTK inhibitors (ibrutinib and zanubrutinib), anti-CD20 antibodies (rituximab), chemotherapeutic drugs, or combinations thereof (14,15). However, the infiltration of BM by malignant lymphoplasmacytic cells and crosstalk with the BM microenvironment is thought to strongly contribute to pathogenesis and resistance to therapy in WM (23-25,28). It is therefore important to develop a therapeutic strategy that also disrupts the BM microenvironment. In this study, we aimed to decipher the crosstalk between WM cells and BMSCs in an in vitro coculture model. We also aimed to explore the effects of a novel combination therapy utilizing mavorixafor, an orally available CXCR4 antagonist, with various B-cell-targeted inhibitors to target WM cells in the BM milieu.

IL-6, an important cytokine that is mainly secreted by stromal cells in the tumor microenvironment, plays a key role in promoting proliferation, angiogenesis, metastasis, and drug resistance of various malignant cells, including DLBCL, MM, and MCL (33,44,45). In patients with WM, IL-6 levels are elevated in the BM and serum, and this increase is associated with increased IgM secretion by WM cells (22,25). Blockage of IL-6R by tocilizumab reduces IgM secretion and tumor growth in a WM mouse xenograft model (26). Here, we provide evidence that BMSCs upregulate IL-6 secretion when cocultured with WM cells. This interaction enhances CXCR4 cell surface expression in WM cells through JAK-STAT3 signaling, ultimately causing increased adhesion to the BMSCs and increased IgM secretion. Finally, we directly link CXCR4 cell surface upregulation in WM cells to resistance to multiple B-cell-targeted inhibitors-those in use (BTK inhibitors ibrutinib and zanubrutinib) and those being studied for the treatment of WM (BTK inhibitors evobrutinib, pirtobrutinib, and nemtabrutinib; BCL-2 inhibitor venetoclax).

IL-6 was previously suggested to boost CXCR4 cell surface expression and increase CXCL12-driven cell migration in astroglia (46). In the context of WM, publicly available data sets confirm increased CXCR4 expression in BM-derived B-cells from patients with WM vs peripheral B-cells from healthy donors. Experimental overexpression of CXCR4 in WM cell lines increased tumor infiltration into BM and other organs, accelerated disease progression, decreased survival, and increased IgM secretion upon transplantation into severe combined immunodeficient (SCID)/beige (Bg) mice (41). Conversely, monoclonal anti-CXCR4 antibody ulocuplumab reduced tumor infiltration in BM, spleen, and lymph nodes in a mouse xenograft model with human WM cells (41). Furthermore, similar in vitro cocultures using BMSCs and non-Hodgkin lymphoma or CML cells also showed increased CXCR4 surface expression, promoting growth, migration, and adhesion of malignant cells to BMSCs as well as resistance to anti-cancer drugs (31,40). Besides its role in promoting the adhesion of WM cells to BMSCs, increased CXCR4 cell surface expression in WM cells may prolong and sustain intracellular signaling via its ligand CXCL12, which is constitutively secreted by BMSCs. This intracellular signaling may promote the drug resistance observed in our WM cells-BMSC coculture model. Supporting this assumption, CXCL12 was previously shown to enhance and sustain extracellular signal-regulated kinase and PI3K-Akt activation in WM cells expressing CXCR4^{WHIM} and protect cells against apoptosis caused by various anticancer drugs (i.e., ibrutinib, bendamustine, fludarabine, bortezomib, and idelalisib) (11,12). Complex crosstalk of the CXCL12/CXCR4 axis with other intracellular signaling pathways also promoted drug resistance in numerous cancers (47). Collectively, our data underline the tight connection between WM cells and BMSCs and its importance in cell adhesion, IgM secretion, and resistance to therapeutic agents. These results also suggest that disruption of the WM cells-BMSC interaction—for example, by targeting the CXCR4/CXCL12 axis and disrupting the protective BM niche—is a promising therapeutic strategy for treating WM.

To test if targeting the CXCR4/CXCL12 axis disrupts the communication between WM cells and BMSCs to overcome drug resistance, we combined different B-cell-targeted inhibitors with mavorixafor in the presence and absence of BMSCs. Mavorixafor, a first-in-class, orally available small-molecule antagonist of CXCR4, is currently being studied in clinical trials for the treatment of patients with WHIM syndrome, WM, and SCN/CN. Our data showed that mavorixafor as a single agent directly induced apoptosis in WM cells and combining mavorixafor with B-cell-targeted inhibitors synergistically induced apoptosis in WM cells, likely by inducing PARP and caspase-3. This synergy may be because these drugs target distinct pathways in WM cells: B-cell-targeted inhibitors used in our assay target BTK or BCL-2, while mavorixafor targets CXCR4. In line with our observation, high-affinity CXCR4 antagonist (BKT140) or monoclonal antibody anti-CXCR4 (ulocuplumab) directly inhibits proliferation, increases apoptosis, and enhances antitumor activity of several anticancer drugs in various lymphoma/leukemia cell lines (39-41). Our data provide evidence that blocking the CXCR4/CXCL12 axis with mavorixafor is an effective way to overcome BMSC-induced resistance to B-cell-targeted inhibitors and to target WM in the BM niche. Mechanistically, mavorixafor blocked the CXCL12-induced calcium mobilization, homing of WM cells to CXCL12-secreted BMSCs, and adhesion of WM cells to BMSCs; it is also likely that mavorixafor enhanced PARP and caspase-3 cleavage caused by B-cell-targeted inhibitors in the presence of BMSCs. In contrast to WM cells, BMSCs were less sensitive to mavorixafor treatment, suggesting a potential therapeutic utility of mavorixafor in WM.

Mavorixafor is well tolerated, with no treatment-related serious adverse events in patients with WHIM syndrome (NCT03005327). Mavorixafor was also reported to block stromal-induced migration of ALL cells, disrupt preestablished adhesion to stroma, and increase sensitivity to chemotherapeutic drugs (vincristine) and targeted therapy (nilotinib) (20). BMSC-derived CXCL12 was shown to activate adhesion-related signaling (e.g., focal adhesion kinase [FAK], proto-oncogene non-receptor tyrosine [SRC] kinase) and enhance the expression of adhesion molecules (e.g., α4β1 integrins) in neoplastic and normal hematopoietic stem cells, facilitating homing and adhesion to BMSCs (21,48,49). Future studies are required to address whether mavorixafor inhibits BMSC-mediated upregulation of adhesion molecules, reducing their adhesion to BMSCs, and sensitizing them to therapeutic agents.

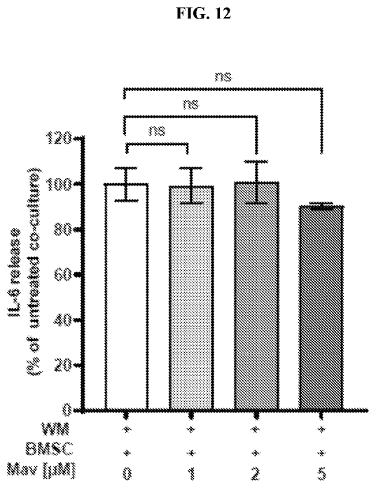

Since overproduction of IgM is a hallmark of WM (1,3), we investigated the effect of mavorixafor as a single agent or in combination with B-cell-targeted inhibitors on IgM secretion. In these studies, mavorixafor inhibited BMSC-induced IgM hypersecretion and synergized with tested therapeutic agents. In previous studies, IL-6 levels were elevated in the sera and BM of patients with WM, and it facilitated IgM secretion in WM cells (22,25). However, addition of mavorixafor had no significant effect on IL-6 release in our WM cell-BMSC coculture model (FIG. 12), suggesting that mavorixafor inhibited IgM hypersecretion independently of IL-6, potentially by disrupting cell-cell adhesion. BMSCs activated STAT3 signaling directly via cell-cell adhesion or indirectly through secreted factors (e.g., IL-6 or IL-21), facilitating IgM secretion in WM cells (21,25,28). Future experiments will have to resolve the question whether mavorixafor reduces IgM hypersecretion via interference with BMSC-induced STAT3 activation.

Collectively, our data show that mavorixafor has several effects in WM cells: (1) mavorixafor synergizes with B-cell-targeted inhibitors to enhance apoptosis, (2) disrupts the crosstalk between WM cells and BMSCs and restores the sensitivity of WM cells to B-cell-targeted inhibitors, and (3) inhibits BMSC-induced IgM hypersecretion in WM and synergizes with B-cell-targeted drugs in this context. Our data provide strong experimental support for the potential use of mavorixafor as a single agent or in combination with B-cell-targeted drugs for the treatment of WM and possibly for other malignancies. Supporting this conclusion, cotreatment with mavorixafor and nilotinib prolonged the survival of C57BL6 mice transplanted with mouse Bcr/Abl⁺ ALL cells (20). Similarly, a combination of mavorixafor and vincristine significantly reduced the number of leukemic cells at extramedullary sites in a mouse xenograft model of a human ALL cells (20). A clinical trial evaluating the efficacy of mavorixafor in combination with ibrutinib in patients with WM with MYD88^{L265P} and CXCR4^{WHIM} mutations is currently ongoing (NCT04274738).

### Example 2: Testing of Double Triple Therapies

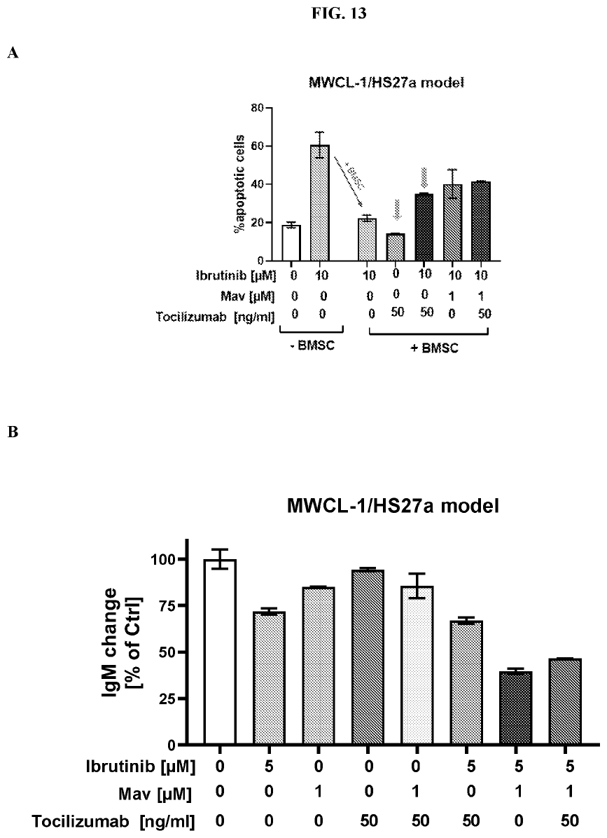

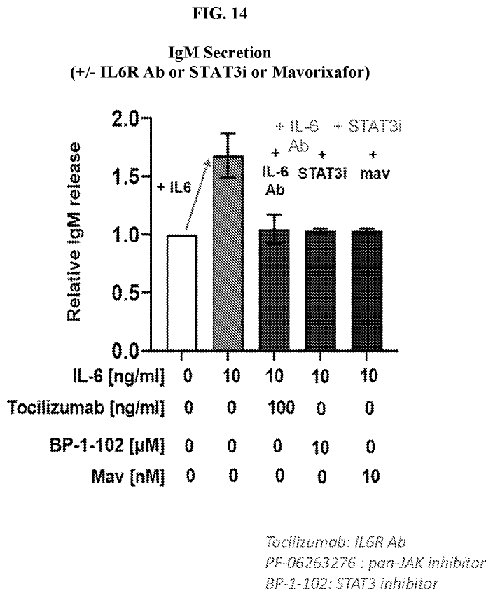

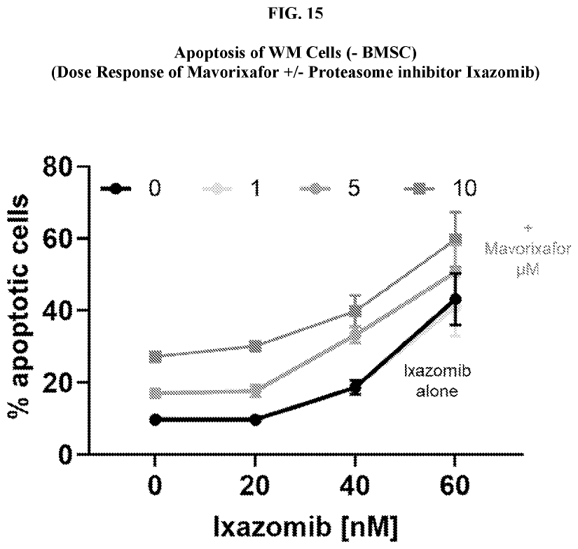

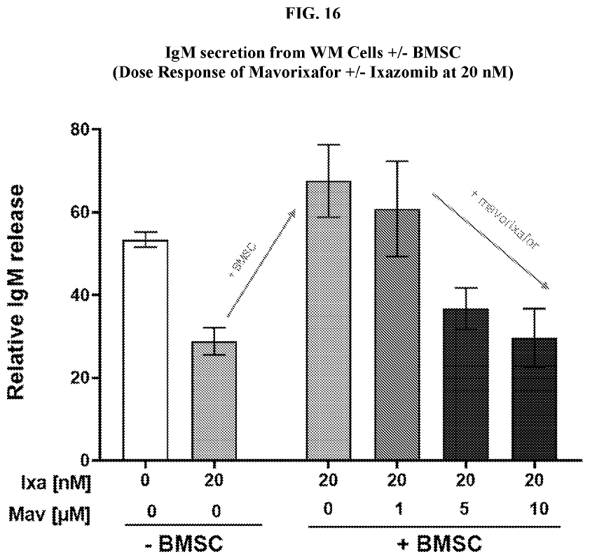

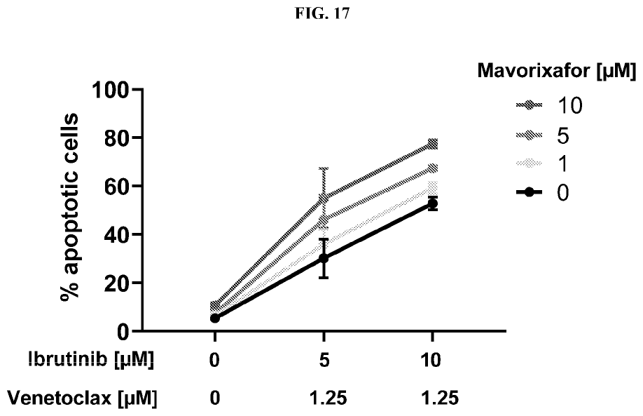

Double and triple combination therapies were tested in a Waldenström's macroglobulinemia (WM) cell line with and without the presence of bone marrow stroma cells (BMSC). As demonstrated in FIG. 13A, in the absence of BMSC, adding Ibrutinib increased the % apoptotic cells from approximately 20% to approximately 60%. However, upon addition of BMSC, neither Ibrutinib nor Tocilizumab as a single therapy increased the percentage of apoptotic cells. However, a double combination therapy of Ibrutinib and Tocilizumab increased the % apoptotic cells to approximately 35%, and a triple combination therapy of Ibrutinib, Tocilizumab, and Mavorixafor increased the % apoptotic cells to approximately 40%. As demonstrated in FIG. 13B, in the presence of BMSC, a triple combination therapy of Ibrutinib, Tocilizumab, and mavorixafor reduced the IgM change compared to single agent therapy with each of Ibrutinib, Tocilizumab, or mavorixafor.

### REFERENCES

1. Advani et al. Updates in prognostication and treatment of Waldenström's macroglobulinemia. Hematol Oncol Stem Cell Ther 2019; 12:179-88.
2. Gertz Waldenström macroglobulinemia: 2021 update on diagnosis, risk stratification, and management. Am J Hematol 2021; 96:258-69.
3. Treon, et al. Genomic landscape of Waldenström macroglobulinemia and its impact on treatment strategies. J Clin Oncol 2020; 38:1198-208.
4. Treon et al. MYD88 L265P somatic mutation in Waldenström's macroglobulinemia. N Engl J Med 2012; 367:826-33.
5. Pal et al. Role of Bruton's tyrosine kinase in B cells and malignancies. Mol Cancer 2018; 17:57.
6. Hunter et al. The genomic landscape of Waldenstrom macroglobulinemia is characterized by highly recurring MYD88 and WHIM-like CXCR4 mutations, and small somatic deletions associated with B-cell lymphomagenesis. Blood 2014; 123:1637-46.
7. Poulain et al. Genomic landscape of CXCR4 mutations in Waldenström macroglobulinemia. Clin Cancer Res 2016; 22:1480-8.
8. Xu et al. Clonal architecture of CXCR4 WHIM-like mutations in Waldenström Macroglobulinaemia. Br J Haematol 2016; 172:735-44.
9. Portella et al., CXCL12 signaling in the tumor microenvironment. Adv Exp Med Biol 2021; 1302:51-70.
10. Santagata et al. CXCR4 and CXCR7 signaling pathways: a focus on the cross-talk between cancer cells and tumor microenvironment. Front Oncol 2021; 11:591386.
11. Cao et al. The WHIM-like CXCR4 (S338X) somatic mutation activates AKT and ERK, and promotes resistance to ibrutinib and other agents used in the treatment of Waldenstrom's Macroglobulinemia. Leukemia 2015; 29:169-76.
12. Cao et al. CXCR4 WHIM-like frameshift and nonsense mutations promote ibrutinib resistance but do not supplant MYD88 (L265P)-directed survival signalling in Waldenström macroglobulinaemia cells. Br J Haematol 2015; 168:701-7.
13. Castillo et al. CXCR4 mutations affect presentation and outcomes in patients with Waldenström macroglobulinemia: A systematic review. Expert Rev Hematol 2019; 1): 873-81.
14. Grimont et al. Current and emerging treatments for Waldenström macroglobulinemia. Acta Haematol 2021; 144:146-57.
15. Ntanasis-Stathopoulos et al. Current and novel BTK inhibitors in Waldenström's macroglobulinemia. Ther Adv Hematol 2021; 12:2040620721989586.
16. An et al. A phase II trial of the Bruton tyrosine-kinase inhibitor zanubrutinib (BGB-3111) in patients with relapsed/refractory Waldenström macroglobulinemia. Clin Cancer Res 2021; 27:5492-501.
17. Castillo et al. Long-term follow-up of ibrutinib monotherapy in treatment-naive patients with Waldenstrom macroglobulinemia. Leukemia 2022; 36:532-9.
18. Castillo et al. Response and survival predictors in a cohort of 319 patients with Waldenström macroglobulinemia treated with ibrutinib monotherapy. Blood Adv 2022; 6:1015-24.
19. Trotman et al. Single-agent ibrutinib for rituximab-refractory Waldenström macroglobulinemia: final analysis of the substudy of the phase III Innovate™ trial. Clin Cancer Res 2021; 27:5793-800.
20. Parameswaran et al., Combination of drug therapy in acute lymphoblastic leukemia with a CXCR4 antagonist. Leukemia 2011; 25:1314-23.
21. Azab et al. FGFR3 is overexpressed waldenstrom macroglobulinemia and its inhibition by Dovitinib induces apoptosis and overcomes stroma-induced proliferation. Clin Cancer Res 2011; 17:4389-99.
22. Elsawa and Ansell. Cytokines in the microenvironment of Waldenström's macroglobulinemia. Clin Lymphoma Myeloma 2009; 9:43-5.
23. Jalali and Ansell Role of the bone marrow niche in supporting the pathogenesis of lymphoid malignancies. Front Cell Dev Biol 2021; 9:692320.
24. Ngo et al. Src tyrosine kinase regulates adhesion and chemotaxis in Waldenstrom macroglobulinemia. Clin Cancer Res 2009; 15:6035-41.
25. Elsawa S F, Novak A J, Ziesmer S C, Almada L L, Hodge L S, Grote D M, et al. Comprehensive analysis of tumor microenvironment cytokines in Waldenstrom macroglobulinemia identifies CCL5 as a novel modulator of IL-6 activity. Blood 2011; 118:5540-9.
26. Han et al. Targeting IL-6 receptor reduces IgM levels and tumor growth in Waldenström macroglobulinemia. Oncotarget 2019; 10:3400-7.
27. Jackson, et al. Modulation of the IL-6 receptor a underlies GLI2-mediated regulation of Ig secretion in Waldenström macroglobulinemia cells. J Immunol 2015; 195:2908-16.
28. Hodge et al. IL-21 in the bone marrow microenvironment contributes to IgM secretion and proliferation of malignant cells in Waldenstrom macroglobulinemia. Blood 2012; 120:3774-82.
29. Kaushal et al. Aberrant extrafollicular B cells, immune dysfunction, myeloid inflammation, and MyD88-mutant progenitors precede Waldenstrom macroglobulinemia. Blood Cancer Discov 2021; 2:600-15.
30. Azab et al. CXCR4 inhibitor AMD3100 disrupts the interaction of multiple myeloma cells with the bone marrow microenvironment and enhances their sensitivity to therapy. Blood 2009; 113:4341-51.
31. Jin et al. CXCR4 up-regulation by imatinib induces chronic myelogenous leukemia (CML) cell migration to bone marrow stroma and promotes survival of quiescent CML cells. Mol Cancer Ther 2008; 7:48-58.
32. Stamatopoulos et al. AMD3100 disrupts the cross-talk between chronic lymphocytic leukemia cells and a mesenchymal stromal or nurse-like cell-based microenvironment: pre-clinical evidence for its association with chronic lymphocytic leukemia treatments. Haematologica 2012; 97:608-15.
33. Zhong, et al. Human bone marrow-derived mesenchymal stem cells promote the growth and drug-resistance of diffuse large B-cell lymphoma by secreting IL-6 and elevating IL-17A levels. J Exp Clin Cancer Res 2019; 38:73.
34. Hunter et al. Transcriptome sequencing reveals a profile that corresponds to genomic variants in Waldenström macroglobulinemia. Blood 2016; 128:827-38.
35. Kaiser et al. Targeting of CXCR4 by the naturally occurring CXCR4 antagonist EPI-X4 in Waldenström's macroglobulinemia. Cancers (Basel) 2021; 13 (4)
36. Chou. Drug combination studies and their synergy quantification using the Chou-Talalay method. Cancer Res 2010; 70:440-6.
37. Nguyen et al. IL2RA promotes aggressiveness and stem cell-related properties of acute myeloid leukemia. Cancer Res 2020; 80:4527-39.
38. Hatzimichael et al. Serum levels of IL-6 and its soluble receptor (sIL-6R) in Waldenström's macroglobulinemia. Eur J Haematol 2001; 66:1-6.
39. Beider, et al. Combination of imatinib with CXCR4 antagonist BKT140 overcomes the protective effect of stroma and targets CML in vitro and in vivo. Mol Cancer Ther 2014; 13:1155-69.
40. Beider et al. Targeting the CD20 and CXCR4 pathways in non-hodgkin lymphoma with rituximab and high-affinity CXCR4 antagonist BKT140. Clin Cancer Res 2013; 19:3495-507.
41. Roccaro et al. C1013G/CXCR4 acts as a driver mutation of tumor progression and modulator of drug resistance in lymphoplasmacytic lymphoma. Blood 2014; 123:4120-31.
42. Chen et al. ROS-induced CXCR4 signaling regulates mantle cell lymphoma (MCL) cell survival and drug resistance in the bone marrow microenvironment via autophagy. Clin Cancer Res 2016; 22:187-99.
43. Agle et al. Calcium mobilization triggered by the chemokine CXCL12 regulates migration in wounded intestinal epithelial monolayers. J Biol Chem 2010; 285:16066-75.
44. Harmer et al. Interleukin-6 interweaves the bone marrow microenvironment, bone loss, and multiple myeloma. Front Endocrinol (Lausanne) 2018; 9:788.
45. Zhang et al. Role of the microenvironment in mantle cell lymphoma: IL-6 is an important survival factor for the tumor cells. Blood 2012; 120:3783-92.
46. Odemis et al. Interleukin-6 and cAMP induce stromal cell-derived factor-1 chemotaxis in astroglia by up-regulating CXCR4 cell surface expression. Implications for brain inflammation. J Biol Chem 2002; 277:39801-8.
47. Shi et al. The role of the CXCL12/CXCR4/CXCR7 chemokine axis in cancer. Front Pharmacol 2020; 11:574667.
48. Peled et al. The chemokine SDF-1 activates the integrins LFA-1, VLA-4, and VLA-5 on immature human CD34 (+) cells: role in transendothelial/stromal migration and engraftment of NOD/SCID mice. Blood 2000; 95:3289-96.
49. Sanz-Rodríguez et al. Chemokine stromal cell-derived factor-1alpha modulates VLA-4 integrin-mediated multiple myeloma cell adhesion to CS-1/fibronectin and VCAM-1. Blood 2001; 97:346-51.
50. Zou et al. “Targeting STAT3 in Cancer Immunotherapy” Molecular Cancer 2020; 19:145.
51. Zhang et al. “Orally bioavailable small-molecule inhibitor of transcription factor Stat3 regresses human breast and lung cancer xenografts” 2012; PNAS 109:9623-28.
52. Jones et al. “Design and Synthesis of a Pan-Janus Kinase Inhibitor Clinical Candidate” 2017; Med. Chem. 60:767-78.
53. Chen et al. Scientific Reports 2021; 11:6517.
54. Gadina et al. “Translational and clinical advances in JAK-STAT biology: The present and future of jakinibs” J. Leukocyte Biology 2018; 104:499-514.

We claim:

1. A method of treating a B-cell disorder in a patient in need thereof, comprising administering to the patient an effective amount of a targeted B-cell therapy or a pharmaceutically acceptable salt thereof; and an effective amount of an IL-6 modulator or a pharmaceutically acceptable salt thereof; and an effective amount of a CXCR4 inhibitor or a pharmaceutically acceptable salt thereof.

2. The method of claim 1, wherein the B-cell disorder is selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma.

3. The method of claim 1 or claim 2, wherein the targeted B-cell therapy is selected from a BTK inhibitor, a BTK degrader, a BCL-2 inhibitor, a BH3 mimetic, and a proteasome inhibitor.

4. The method of any one of claims 1-3, wherein the targeted B-cell therapy is selected from Ibrutinib; Zanubritinib; Acalubritinib; Evobrutinib; Tirabrutinib; Rilzabrutinib; Tolebrutinib Fenebrutinib; Orelabrutinib; BMS-986195; Branebrutinib, Elsubrutinib, ABBV-105; Remibrutinib; Spebrutinib; Poseltinib; vecabrutinib, LCB 03-0110; LFM-A13; PCI 29732; PF 06465469; (−)-Terreic acid; BMX-IN-1; BI-BTK-1; BMS-986142; CGI-1746; GDC-0834; olmutinib, PLS-123; PRN1008; RN-486; Nemtabrutinib; and Pirtobrutinib, or a pharmaceutically acceptable salt thereof.

5. The method of any one of claims 1-3, wherein the targeted B-cell therapy is selected from NX-2127, MT802, L18I, SPB5208, or RC-1, or a pharmaceutically acceptable salt thereof.

6. The method of any one of claims 1-3, wherein the targeted B-cell therapy is selected from Venetoclax, BGB-11417, LOXO-338, LP-108, S55746, APG-2575, APG-1252, AT-101, TQB3909, obatoclax, GDC-0199, ABT-737, and navitoclax; or a pharmaceutically acceptable salt thereof.

7. The method of any one of claims 1-3, wherein the targeted B-cell therapy is selected from ixazomib; bortezomib; carfilzomib; thalidomide, and everolimus; or a pharmaceutically acceptable salt thereof.

8. The method of any one of claims 1-6, wherein the IL-6 modulator is selected from Tocilizumab; Sarilumab; Satralizumab; Vobarilizumab; Siltuximab; Olokizumab; Sirukumab; Clazakizumab; Ziltivekimab; NCT03926117; and Avidia; or a pharmaceutically acceptable salt thereof.

9. The method of any one of claims 1-7, wherein the CXCR4 inhibitor is selected from Motixafortide; POL6326; PRX177561; USL311; Burixafor; LY2510924; and PF06747143; or a pharmaceutically acceptable salt thereof.

10. The method of any one of claims 1-7, wherein the CXCR4 inhibitor is selected from mavorixafor; Compound 3: 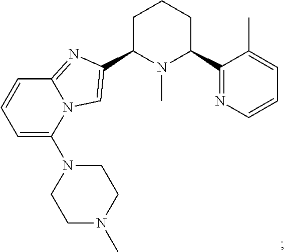 [SMILES: Cc1cccnc1[C@@H]1CCC[C@H](c2cn3c(N4CCN(C)CC4)cccc3n2)N1C]
Compound 1: 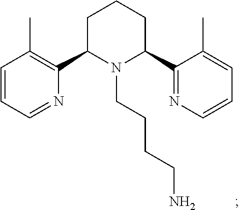 [SMILES: Cc1cccnc1[C@H]1CCC[C@@H](c2ncccc2C)N1CCCCN]
plerixafor; and ulocuplumab; or a pharmaceutically acceptable salt thereof.

11. The method of claim 10, wherein the CXCR4 inhibitor is mavorixafor or a pharmaceutically acceptable salt thereof.

12. The method of claim 1 or claim 2, wherein the targeted B-cell therapy is selected from Ibrutinib; Zanubritinib; and Acalubritinib or a pharmaceutically acceptable salt thereof; and wherein the IL-6 modulator is selected from Tocilizumab; Sarilumab; and Satralizumab; or a pharmaceutically acceptable salt thereof, and the CXCR4 inhibitor is mavorixafor a pharmaceutically acceptable salt thereof.

13. The method of claim 12, wherein the targeted B-cell therapy is Ibrutinib and wherein the the IL-6 modulator is Tocilizumab.

14. The method of claim 12, wherein the CXCR4 inhibitor is mavorixafor or a pharmaceutically acceptable salt thereof.

15. A method of treating a B-cell disorder in a patient in need thereof, comprising administering to the patient about 560 mg once daily Ibrutinib or a pharmaceutically acceptable salt thereof; and an effective amount of an IL-6 modulator or a pharmaceutically acceptable salt thereof; and an effective amount of a CXCR4 inhibitor or a pharmaceutically acceptable salt thereof.

16. A method of treating a B-cell disorder in a patient in need thereof, comprising administering to the patient about 280 mg once daily Ibrutinib or a pharmaceutically acceptable salt thereof; and an effective amount of an IL-6 modulator or a pharmaceutically acceptable salt thereof; and an effective amount of a CXCR4 inhibitor or a pharmaceutically acceptable salt thereof.

17. A method of treating a B-cell disorder in a patient in need thereof, comprising administering to the patient about 140 mg once daily Ibrutinib or a pharmaceutically acceptable salt thereof; and an effective amount of an IL-6 modulator or a pharmaceutically acceptable salt thereof; and an effective amount of a CXCR4 inhibitor or a pharmaceutically acceptable salt thereof.

18. The method of any one of claims 14-17, wherein the B-cell disorder is selected from chronic lymphocytic leukemia (CLL), small lymphocytic lymphoma (SLL), mantle cell lymphoma, marginal zone lymphoma, Waldenström's macroglobulinemia (WM), follicular lymphoma, and diffuse large B-cell lymphoma.

19. The method of any one of claims 14-17, wherein the B-cell disorder is selected from mantle cell lymphoma and marginal zone lymphoma.
